(19) United States
(12) Patent Application Publication
(10) Pub. No.: US 2018/0000973 A1
(43) Pub. Date: Jan. 4, 2018

(54) AGENTS FOR USE IN THE DETECTION OF NUCLEASE ACTIVITY

(71) Applicant: Frank J. HERNÁNDEZ HINCAPÉ, San Sebastian (ES)

(72) Inventors: Frank J. HERNÁNDEZ HINCAPIÉ, San Sebastian (ES); Luiza Iuliana HERNANDEZ, Donostia, San Sebastian, Gipuzkoa (ES)

(21) Appl. No.: 15/520,828

(22) PCT Filed: Oct. 20, 2015
(86) PCT No.: PCT/EP2015/074279

§ 371 Date: Apr. 20, 2017

(30) Foreign Application Priority Data
Oct. 21, 2014 (EP) 14382411.8

(51) Int. Cl. A61K 49/10; A61K 49/08; G01N 33/574

(52) U.S. Cl. CPC A61K 49/10; G01N 33/57438; A61K 49/085; G01N 2333/922

(57) ABSTRACT

The present invention relates to the field of diagnostics and, more in particular, to MRI activatable contrast agents and compositions thereof for the detection of nuclease activity, wherein said nuclease activity is caused by microbial infection or by nuclease activity related to cancer, particularly colon cancer or pancreatic cancer. Activatable contrast agents for MRI have been developed, wherein the oligonucleotide is flanked by a paramagnetic and a superparamagnetic agent, and thus providing magnetic quenching. Moreover, the oligonucleotide has regions that confer resistance to mammalian endonucleases and sensitivity to microbial endonucleases. When the activatable contrast agent of the invention is in the presence of microbial nuclease activity or a tumour cell nuclease activity, the oligonucleotide is cleaved, agents are unquenched, and the signal derived from the activated contrast agent is detected by MRI.

### TECHNICAL FIELD

The present invention relates to the field of diagnostics and, more in particular, to MRI activatable contrast agents and compositions thereof for the detection of nuclease activity, wherein said nuclease activity is caused by microbial infection or by nuclease activity related to cancer, particularly colon cancer or pancreatic cancer. Activatable contrast agents for MRI have been developed, wherein the oligonucleotide is flanked by a paramagnetic and a superparamagnetic agent, and thus providing magnetic quenching. Moreover, the oligonucleotide has regions that confers resistance to mammalian endonucleases and sensitivity to microbial endonucleases. When the activatable contrast agent of the invention is in the presence of microbial nuclease activity or a tumour cell nuclease activity, the oligonucleotide is cleaved, agents are unquenched, and the signal derived from the activated contrast agent is detected by MRI.

### BACKGROUND ART

Magnetic resonance imaging (MRI) is a clinical technique used in radiology to explore the anatomy and function of the body for health and disease. MRI applies a strong magnetic field and radio frequencies to create images of the body. It has become one of the most common tools available for medical diagnosis. The major advantages of MRI in the clinic are the unlimited tissue penetration and the high resolution of the images, which make MRI a leading technology in the clinical field. However, MRI lacks sensitivity to determine biochemical reactions or to diagnose specific human conditions, this being the main drawback of the technology. Thus, the use of contrast agents (CAs) is frequently required to obtain meaningful clinical information.

Contrast agents can be defined as a contrast media to improve the visibility of internal body structures by MRI. MRI contrast agents have made a significant impact in the use of MRI for various clinical indications. Since the introduction of the first MRI contrast agent Gd-DTPA (Magnevist, Schering A G) in 1988, there has been a tremendous increase in the number of contrast-enhanced examinations. MRI contrast agents contain paramagnetic or superparamagnetic metal ions that affect the MRI signal properties of surrounding tissue. These contrast agents are used primarily to increase the sensitivity of MRI for detecting various pathological processes and also for characterizing various pathologies. In addition, the contrast agents are used for depicting normal and abnormal vasculature, or flow-related abnormalities and pathophysiologic processes like perfusion.

Recently, activatable MRI contrast agents have emerged as an alternative to improve MRI technology. Activatable contrast agents report on a change of relaxivity based on a specific stimulus in the surrounding microenvironment, which allows MRI to measure biological reactions. Activatable contrast agents offer interesting features such as low background noise and high sensitivity, which is nicely complemented by MRI. Although several activatable probes for MRI have been described previously, most of them rely on non-specific physical responses to stimuli such as pH and light. Another kind of stimulus that involves enzymes, such as β-galactosidase or β-glucuronidase, has been used as molecular trigger mechanism for MRI detection, however, with limited applicability. Because there is no generic system to develop MRI activatable probes, only few activatable approaches have therefore been incorporated into MRI technology. Thus, a platform with generic capability for the development of MRI activatable probes is highly desirable.

On the other hand, the current standard methods for detecting conditions such as bacterial infections in the clinic are still mainly based on bacterial cultures and biopsy. These two techniques have shown significant limitations. In the case of bacterial cultures, the time is an important factor, since bacterial infections could cause death in even 24 hours, and the currently available clinical diagnosis based on these cultures normally could take from days to weeks. The biopsy is an invasive procedure, not risk-free for the patient and highly trained personnel is needed to carry out the procedure. Moreover, this technique is prone to false negatives. Considering that every year half a million people become infected by Staphylococcus aureus bacteria in the United States, and 20,000 of those who become infected die, early and accurate detection of bacterial infections becomes an important public health issue.

A nuclease-activated probe for non-invasive imaging of Staphylococcus aureus infection has been described (Hernández F J et al. 2014 Nat Med 20: 301-306). The probe is able to detect the activity of the micrococcal nuclease (MN), and comprises an oligonucleotide which is resistant to mammalian nucleases, flanked by a fluorophore and a quencher. However, this probe shows limited tissue penetration.

US2005/0036947 discloses polymeric target-specific activatable polymeric MRI agents, MRI contrast enhancing-agents and MRI methods for detecting specific diseases. These MRI contrast-enhancing agents comprise an extended poly(amino acid) conjugated to chelating moieties that form coordination complexes with paramagnetic ions.

US2014/0044648 describes imaging agents based on activatable probes including a superparamagnetic core and a polymeric matrix coating the metal oxide core. The polymeric matrix is configured to release the paramagnetic agent when subjected to a medium having a pH less than a normal physiological pH.

MRI contrast agents for the sensing of small molecules such as adenosine have also been described (Xu W & Lu Y 2011 Chem Commun 47(17): 4998-5000). These contrast agents are based on a DNA aptamer that is conjugated to a Gd compound and a protein streptavidin. The binding of adenosine to its aptamer results in the dissociation of the Gd compound from the large protein, leading to decreases in the rotational correlation time and thus change of MRI contrast.

Protein-based Gd³⁺ MRI contrast agents (ProCAs) equipped with several desirable capabilities have been proposed for in vivo application of MRI of tumor biomarkers (Xue S et al. 2013 Wiley Interdiscip Rev Nanomed Nanobiotechnol 5(2): 163-179). The design and mechanism of action of Gd-based MRI contrast agents targeted to proteins has been described (Caravan P 2009 Accounts Chem Res 42(7): 851-862).

All in all, there is still a need in the state of the art to develop suitable, effective, and sensitive contrast agents useful in MRI to accurately identify disease conditions such as bacterial infections and tumor development.

### BRIEF SUMMARY OF THE INVENTION

The author of the present invention has developed a contrast agent for use in medical MRI imaging applications, more particularly a MRI activatable probe that works as a contrast agent for detecting nuclease activity, in particular microbial-associated nuclease activity or tumor cell-associated nuclease activity, using chemically modified oligonucleotides as substrates. This contrast agent can be activated by a specific nuclease, such as a microbial nuclease or a tumor cell nuclease, and it can produce measurable MRI signals once the nuclease activation has occurred. As such, this invention aims primarily at the medical diagnostic sector and, in particular, at detecting bacterial infections or tumor cells, particularly colon tumor cells or pancreatic tumor cells, in a subject. MRI activatable contrast agents according to the invention provide a fast (less than 1 hour) specific diagnostic, in contrast to time consuming biopsy-based procedures. MRI activatable contrast agents according to the invention also provide a non-invasive method with confident diagnostic, in contrast to invasive and prone to false negatives procedures based on biopsies. Additionally, MRI activatable contrast agents according to the invention provide a fast and accurate identification of the infection or tumor development loci, providing highly relevant information for the administration of an appropriate treatment to the subject. Furthermore, MRI activatable contrast agents according to the invention have unlimited tissue penetration, excellent spatial resolution (10-100 μm) and MRI devices are approved and commonly used in the clinic. Importantly, this invention overcomes the drawbacks associated with other contrast agents described in the art, such as activatable probes based on fluorescence technology showing limited/poor tissue penetration (approximately 3 cm), poor spatial resolution (1-10 mm), and fluorescence imaging devices which are not yet approved for clinical use (Ahrens ET & Bulte J W M 2013 Nat Rev Immunol 13: 755-763).

Therefore, the new set of MRI activatable contrast agents developed by the inventor of the present invention would facilitate the detection of nuclease activity both in vivo and in vitro, and the diagnosis of human conditions such as bacterial infections or development of tumors at an early stage.

Thus, in a first aspect, the present invention is related to an activatable contrast agent for magnetic resonance imaging (MRI) comprising:

(i) a superparamagnetic agent,

(ii) at least one paramagnetic agent, and

(iii) at least one DNA, RNA or DNA-RNA, single or double stranded oligonucleotide, one end of said at least one oligonucleotide operably linked to the superparamagnetic agent in (i) and the other end of said at least one oligonucleotide operably linked to the at least one paramagnetic agent in (ii), wherein said at least one oligonucleotide comprises
at least one region comprising nucleotides conferring resistance to mammalian endonucleases, and
at least one region comprising a nucleotide sequence conferring sensitivity to a microbial nuclease but not to mammalian endonucleases,

and wherein said oligonucleotide in (iii) allows magnetic quenching between the superparamagnetic agent in (i) and the at least one paramagnetic agent in (ii).

In a further aspect, the invention relates to a pharmaceutical composition comprising the above agent.

In a further aspect, the invention relates to a magnetic resonance imaging (MRI) method for detecting a nuclease activity in a subject, wherein said nuclease activity is a microbial nuclease activity or a tumor cell nuclease activity, that comprises:

(i) administering the activatable contrast agent as above or a pharmaceutical composition as above to said subject, and

(ii) detecting activated contrast agent by MRI.

In a further aspect, the invention relates to an in vitro method for determining whether a subject suffers or not from a microbial infection that comprises

(i) contacting a sample from said subject with a contrast agent as above or with a pharmaceutical composition as above,

(ii) detecting the signal derived from the contrast agent, and

(iii) comparing the signal of the contrast agent detected in (ii) to a reference value,

wherein
if the contrast agent shows a signal detectable by MRI higher than a reference value, then the subject suffers from a microbial infection, or
if the contrast agent shows a signal detectable by MRI similar to or lower than a reference value, then the subject does not suffer from a microbial infection.

In a further aspect, the invention relates to an in vitro method for determining whether a subject suffers or not from colon cancer or from pancreatic cancer that comprises

(i) contacting a sample from said subject with a contrast agent as above or with a pharmaceutical composition as above,

(ii) detecting the signal derived from the contrast agent, and

(iii) comparing the signal of the contrast agent detected in (ii) to a reference value,

wherein
if the contrast agent shows a signal detectable by MRI higher than a reference value, then the subject suffers from colon cancer or from pancreatic cancer, or
if the contrast agent shows a signal detectable by MRI similar to or lower than a reference value, then the subject does not suffer from colon cancer or from pancreatic cancer.

In a last aspect, the invention relates to an in vitro method for detecting nuclease activity in a biological sample that comprises

(i) contacting the sample with a MRI activatable contrast agent as above or with a pharmaceutical composition as above, under suitable conditions for interaction between the sample and the contrast agent,

(ii) detecting the signal derived from the contrast agent, and

(iii) comparing the signal of the contrast agent detected in (ii) to a reference value,

wherein
if the contrast agent shows a signal detectable by MRI higher than a reference value, then nuclease activity exists in the sample, or
if the contrast agent shows a signal detectable by MRI similar to or lower than a reference value, then nuclease activity does not exist in the sample, or that, alternatively, comprises

(i′) contacting the sample with a MRI activatable contrast agent according to the invention or with a pharmaceutical composition according to the invention, under suitable conditions for interaction between the sample and the contrast agent, and

(ii′) determining whether a signal from the activated contrast agent is detected,

wherein detection of the activated contrast agent is indicative of nuclease activity in the biological sample, and absence of detection of the activated contrast agent is indicative of absence of nuclease activity in the biological sample.

### BRIEF DESCRIPTION OF THE FIGURES

FIG. 1 shows a scheme of a magnetic resonance image (MRI) activatable contrast agent according to the invention. A) MRI activatable contrast agent, composed of Iron Oxide Nanoparticles (ION) conjugated with a gadolinium modified-oligonucleotide. Gadolinium (Gd) is quenched at this initial state (“off”) by the proximity to the ION. B)

Upon oligonucleotide degradation by micrococcal nuclease (MN), C) gadolinium diffuses away from the ION and it recovers its relaxation properties (unquenching).

FIG. 2 shows the secondary structure of the oligonucleotides of sequences SEQ ID NO: 1-3 comprised by the contrast agent of the invention. Deoxythymidines (T) and deoxyadenosines (A) are shown inside circles. All other nucleotides are 2′-O-methyl modified nucleotides. Watson-Crick base pairings are represented by a line intercepted by a circle.

FIG. 3 shows the evaluation of relaxation properties of a MRI activatable contrast agent according to the invention which comprises an oligonucleotide of SEQ ID NO:1 under physio logical conditions. The oligonucleotide 5′NH2-mCmUmCmGTTmCmGmUmUmC-Biotin-3′ [5′NH2-(SEQ ID NO: 1)-Biotin-3′]modified with Gd(Gd-SEQ#1) was measured as positive control of Gd signal, reporting a T1 value of 1211 ms (black squares). Then, MRI-SEQ#1 probe (Gd-SEQ#1-ION) was evaluated in the absence and presence of nucleases (MN). The MRI-SEQ#1 probe incubated in PBS only has reported significant stability (1 hour) and a T1 value of 2308 ms under physiological conditions. In contrast, the MRI-SEQ#1 probe incubated with PBS containing MN under the same conditions, has shown a T1 value of 1179 ms (triangles, dashed line). All the measurements were carried out in triplicate; the results show average relaxation intensity and the error bars represent standard deviations. au, arbitrary units.

FIG. 4 shows the evaluation of relaxation properties of a MRI activatable contrast agent according to the invention which comprises an oligonucleotide of SEQ ID NO:2 under physio logical conditions. The oligonucleotide 5′NH2-TTmCmGmCmUmUmCmGmGmCmGmAmA-Biotin-3′ [5′NH2-(SEQ ID NO: 2)-Biotin-3′]modified with Gd(Gd-SEQ#2) was measured as positive control of Gd signal, reporting a T1 value of 1298 ms (black squares). Then, MRI-SEQ#2 probe (Gd-SEQ#2-ION) was evaluated in the absence and presence of nucleases (MN). The MRI-SEQ#2 probe incubated in PBS only has reported significant stability (1 hour) and a T1 value of 2196 ms under physiological conditions. In contrast, the MRI-SEQ#2 probe incubated with PBS containing MN under same conditions, has shown a T1 value of 1357 ms (triangles, dashed line). All the measurements were carried out in triplicate; the results show average relaxation intensity and the error bars represent standard deviations.au, arbitrary units.

FIG. 5 shows the evaluation of relaxation properties of a MRI activatable contrast agent according to the invention which comprises an oligonucleotide of SEQ ID NO:3 under physio logical conditions. The oligonucleotide 5′NH2-mCTAmCmGmCmUmUmCmGmGmCmGTAmG-Biotin-3′ [5′NH2-(SEQ ID NO: 3)-Biotin-3′] modified with Gd(Gd-SEQ#3) was measured as positive control of Gd signal, reporting a T1 value of 1286 ms (black squares). Then, MRI-SEQ#3 probe (Gd-SEQ#3-ION) was evaluated in the absence and presence of nucleases (MN). The MRI-SEQ#3 probe incubated in PBS only has reported significant stability (1 hour) and a T1 value of 2077 ms under physiological conditions. In contrast, the MRI-SEQ#3 probe incubated with PBS containing MN under same conditions has shown a T1 value of 1389 ms (triangles, dashed line). All the measurements were carried out in triplicate; the results show average relaxation intensity and the error bars represent standard deviations.au, arbitrary units.

FIG. 6 shows a scheme of the synthesis process of the MRI probe. First, the oligonucleotide is modified at the 5′-end with a DTPA chelator, that allows for efficient attachment of the Gadolinium (Gd+3). Then, the 3′-end of the oligonucleotide, that was modified during the synthesis with a thiol group (SH), is coupled to the maleimide group present on the surface of the iron oxide nanoparticles (ION).

FIG. 7 shows the activatable dual-mode (T1 and T2) MRI contrast agent for the specific targeting of bacteria. MRI signals for A. T1 and B. T2 were acquired for bacterial cultures of S. aureus (bacteria target), S.epidermidis (non-specific bacteria), along with culture media (TSB) and pig serum as additional controls. In both cases, T1 and T2 respectively, the MRI-probe was able to identify S. aureus as the targeted bacteria.

FIG. 8 shows the activation of the AAA chimeric probe (synthesized using the MRI-approach ION-probe-Gd+3)by a specific cancer marker nuclease (SND1) The relaxivity values were recorded.

FIG. 9 shows the activation of the all 2′-Fluoro probe (synthesized using the MRI-approach tION-probe-Gd+3) by pancreatic cancer cell lysates. The relaxivity values were recorded.

### DETAILED DESCRIPTION OF THE INVENTION

Definitions

The term “chemically modified nucleotide”, as used herein, or simply “modified nucleotide”, relates to oligonucleotides with a structure conferring resistance to an exonuclease and/or an endonuclease, preferably to mammalian nucleases. This term encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose. Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylamino methyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine;uracil-5-oxyacetic acid methyl ester; pseudouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; 2,6,-diaminopurine;methylpseudouracil; 1-methylguanine; 1-methylcytosine.

The term “contrast agent”, as used herein, also known as “imaging agent” or “contrast probe” relates to a biocompatible compound the use of which facilitates the differentiation of different parts of the image, by increasing the “contrast” between those different regions of the image. Contrast agents are used to improve the visibility of internal body structures. In the context of the invention, the contrast agent is a contrast agent for magnetic resonance image (MRI). MRI contrast agents alter the relaxation times of atoms within body tissues where they are present after oral or intravenous administration. In MRI scanners sections of the body are exposed to a very strong magnetic field, then a radio frequency pulse is applied causing some atoms (including those in contrast agents) to spin and then relax after the pulse stops. This relaxation emits energy which is detected by the scanner and is mathematically converted into an image. The MRI image can be weighted in different ways giving a higher or lower signal. Contrast agents for magnetic resonance imaging include gadolinium chelates, manganese chelates, chromium chelates and iron particles. MRI contrast agents can include complexes of metals selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

The term “decreased signal” or “lower signal”, as used in the present invention in relation to the signal of the contrast agent detected by MRI, relates to a situation where the contrast agent signal is decreased at least 5%, at least 10%, at least 20%, at least 30%, at least 40%, at least 50%, at least 60%, at least 70%, at least 80%, at least 90% or at least 100% when compared to the corresponding reference value or control.

The term “increased signal” or “higher signal”, as used in the present invention in relation to the signal of the contrast agent detected by MRI, relates to a situation where the contrast agent signal is increased at least 5%, at least 10%, at least 20%, at least 30%, at least 40%, at least 50%, at least 60%, at least 70%, at least 80%, at least 90% or at least 100% when compared to the corresponding reference value or control.

The term “infection”, as used herein, relates to invasion by bacteria, viruses, fungi, protozoa or other microorganisms, referring to the undesired proliferation or presence of invasion of pathogenic microbes in a host organism. It includes the excessive growth of microbes that are normally present in or on the body of a mammal or other organism. More generally, a microbial infection can be any situation in which the presence of a microbial population(s) is damaging to a host mammal. Thus, a microbial infection exists when excessive numbers of a microbial population are present in or on the body of a mammal, or when the effects of the presence of a microbial population(s) is damaging the cells or other tissue of a mammal. In a particular embodiment, the infection is a bacterial infection, a viral infection or a fungal infection. In a more particular embodiment, the infection is a bacterial infection.

The term “magnetic quenching”, as used herein, relates to the termination of the magnet operation that occurs when the superparamagnetic agent (e.g. iron oxide core) will affect the relaxation properties of the paramagnetic agent (e.g. Gd-DTPA), resulting in the quenching of the T1 signal.

The term “magnetic resonance image”, as used herein, abbreviated as MRI, also known as magnetic resonance imaging, or magnetic resonance tomography (MRT), relates to a medical imaging technique most commonly used in radiology to visualize the structure and function of the body. It provides detailed images of the body in any plane. MRI uses non ionizing radiation, but uses a powerful magnetic field to align the nuclear magnetization of (usually) hydrogen atoms in water in the body. Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body. When a subject lies in a scanner, the hydrogen nuclei (i.e., protons) found in abundance in an animal body in water molecules, align with the strong main magnetic field. A second electromagnetic field that oscillates at radio frequencies and is perpendicular to the main field, is then pulsed to push a proportion of the protons out of alignment with the main field. These protons then drift back into alignment with the main field, emitting a detectable radio frequency signal as they do so. Since protons in different tissues of the body (e.g., fat versus muscle) realign at different speeds, the different structures of the body can be revealed. The term “microorganism”, as used herein, also known as microbe, relates to bacteria, viruses, and fungi. Microorganims causing a disease in the host in which they reside are pathogenic microorganisms or pathogens.

The term “nuclease”, as used herein, relates to an enzyme capable of cleaving the phosphodiester bonds between the nucleotide subunits of nucleic acids. Nucleases are classified as endonucleases and exonucleases. Nucleases according to the invention include, without limitation, deoxyribonucleases and ribonucleases. The term “endonuclease” relates to a nuclease enzyme cleaving the phosphodiester bond within a polynucleotide chain. Endonucleases include, without limitation, restriction endonucleases, which are restriction enzymes cleaving only at very specific nucleotide sequences. The term “exonuclease” relates to a nuclease enzyme cleaving the phosphodiester bond between nucleotide at the end of the polynucleotide chain. Exonucleases include, without limitation, 5′ to 3′ exonucleases, 3′ to 5′ exonucleases and poly(A)-specific 3′ to 5′ exonucleases. The term “microbial nuclease” relates to nucleases of any microorganism, including bacteria, fungi, and virus. The term “mammalian nuclease” relates to a nuclease of any mammal. Particularly preferred microbial nucleases according to the invention include the micrococcal nuclease (MN) from Staphylococcus aureus, the endA nuclease from Streptococcus pneumoniae, the endonuclease IV from Mycobacterium tuberculosis, the UL98 nuclease from cytomegalovirus, and nuclease S1 from Aspergillus.

The term “nucleic acid” relates to a deoxyribonucleotide or ribonucleotide polymer in either single or double stranded form and, unless otherwise limited, encompasses natural nucleotides and analogues of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in an oligonucleotide or in a polynucleotide. A “nucleotide sequence” or “nucleic acid sequence” refers to the sequence of bases in an oligonucleotide or in a polynucleotide.

The term “oligonucleotide”, as used herein, relates to a single or double stranded, DNA or RNA molecule, with up to 50, 45, 40, 35, 30, 25, 20, 19, 18, 17, 16, 15, 14 or 13 bases in length (upper limit). The oligonucleotides of the invention are preferably DNA or RNA molecules of at least 2, at least 3, at least 4, at least 5, at least 6, at least 7, at least 8, at least 9, at least 10, at least 11, at least 12, or 13 bases in length (lower limit). Ranges of base lengths can be combined in all different manners using the afore-mentioned lower and upper limits, for example at least 2 and up to 50 bases, at least 8 and up to 25 bases, at least 5 and up 15 bases or at least 8 and up to 18 bases.

The term “operably linked”, as used herein, refers to the association of two chemical moieties so that the function of one is affected by the other, e.g., an arrangement of elements wherein the components so described are configured so as to perform their usual function. In the context of the invention, it relates to the functional relation and contiguous location of the elements of the activatable contrast agent for MRI according to the invention, wherein one end of the oligonucleotide is operably linked to the paramagnetic agent and the other end of said oligonucleotide is operably linked to the superparamagnetic agent.

The term “paramagnetic agent”, as used herein, relates to an agent used to enhance MRI images and display areas of hypervascularity and associated pathology. They have their strongest effect in T1 (spin-lattice relaxation time) weighted imaging because they predominantly alter the T1 relaxation time in the tissues in which they have accumulated. Paramagnetic materials are metals with unpaired electrons in the outer orbital shells (transition and lanthanide metals), giving rise to magnetic dipoles when exposed to a magnetic field. Since the magnetic moment of an electron is about 700 times larger than that of a proton (due to smaller mass), the paramagnetic ions induce large fluctuating magnetic fields experienced by nearby protons. If the frequency of this fluctuation has a component close to the Larmor frequency it will result in a significant enhancement of proton relaxation. Paramagnetic agents for MRI include, without limitation, gadolinium (Gd)-based agents and manganese (Mn)-based agents.

The term “pharmaceutical composition”, as used herein, relates to a composition comprising a detection effective amount of the activatable contrast agent according to the present invention and at least one pharmaceutically acceptable excipient or carrier. The terms “pharmaceutically acceptable excipient”, or “pharmaceutically acceptable carrier”, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed, and is compatible with other ingredients of the formulation. Suitable carriers include, but are not limited to water, dextrose, glycerol, saline, ethanol, and combinations thereof. The carrier can contain additional agents such as wetting or emulsifying agents, pH buffering agents, or adjuvants, which enhance the effectiveness of the formulation. Adjuvants could be selected from the group consisting of sterile liquids, such as water and oils, including those of petroleum, animal, vegetable, or synthetic origin, such as peanut oil, soybean oil, mineral oil, sesame oil, and similars. Water or saline aqueous solutions and aqueous dextrose and glycerol solutions, particularly for injectable solutions, are preferably used as vehicles.

Suitable pharmaceutical vehicles are described in “Remington's Pharmaceutical Sciences” by E. W. Martin, 22^{th} Edition, 2012.

The term “reference value” or “reference level”, as used herein, relates to a laboratory value used as a reference for the expression level values/data obtained from samples to be analyzed or from samples from subjects to be diagnosed with a disease or disorder.

The term “sample” or “biological sample”, as used herein, refers to biological material isolated from a subject. The biological sample contains any biological material suitable for detecting a nuclease activity, particularly a microbial nuclease activity or a tumor cell nuclease activity. The biological sample can comprise cell and/or non-cell material of the subject. The sample can be isolated from any suitable tissue or biological fluid such as, for example blood, saliva, plasma, serum, urine, cerebrospinal liquid (CSF), feces, a surgical specimen, a specimen obtained from a biopsy, and a tissue sample embedded in paraffin.

The term “secondary structure”, as used herein in relation to a nucleotide sequence, relates to the base-pairing interactions within a single molecule or set of interacting molecules, and can be represented as a list of bases which are paired in a nucleic acid molecule. The secondary structures in DNA molecules mostly exist as fully base-paired double helices, while biological secondary structures in RNA are usually single stranded and often form complicated base-pairing interactions due to its increased ability to form hydrogen bonds stemming from the extra hydroxyl group in the ribose sugar. Nucleic acids secondary structures include helices (contiguous base pairs), and various kinds of loops (unpaired nucleotides surrounded by helices). Frequently these elements, or combinations of them, can be further classified, for example, tetraloops, pseudoknots, and stem-loops.

The term “subject” or “individual” or “animal” or “patient” or “mammal,” is meant any subject, particularly a mammalian subject, for whom diagnosis or prognosis is desired. Mammalian subjects include humans, domestic animals, farm animals, and zoo, sports, or pet animals such as dogs, cats, guinea pigs, rabbits, rats, mice, horses, cattle, cows, and so on. In a preferred embodiment of the invention, the subject is a mammal. In a more preferred embodiment of the invention, the subject is a human.

The term “superparamagnetic agent”, as used herein, relates to agents based on magnetite (Fe₃O₄) or maghemite (γ-Fe₂O₃) water insoluble iron oxide crystals with a core diameter in the range 5-10 nm. These crystals are often referred to as nanoparticles, and each nanoparticle contains several thousand paramagnetic Fe ions (Fe²⁺ and Fe³⁺). If the Fe ions are magnetically ordered within the crystal, the net magnetic moment of the nanoparticle is so large that it greatly exceeds that of typical paramagnetic ions. This effect is referred to as superparamagnetism and is characterized by a large magnetic moment in the presence of an external magnetic field but no remnant magnetic moment when the field is zero. Superparamagnetic agents can also induce strong enhancement of the T₁-relaxation rate of water (depending on size and composition of the particles), but their dominant effect is on T2/T2* relaxation due to the large magnetic moment of the nanoparticles.

Superparamagnetic agents for MRI include, without limitation, iron platinum-based agents and iron oxide-based agents.

The term “tumor”, as used herein, also known as “cancer” or “tumor disease”, refers to a broad group of diseases involving unregulated cell growth and which are also referred to as malignant neoplasms. The term is usually applied to a disease characterized by uncontrolled cell division (or by an increase of survival or apoptosis resistance) and by the ability of said cells to invade other neighbouring tissues (invasion) and spread to other areas of the body where the cells are not normally located (metastasis) through the lymphatic and blood vessels, circulate through the bloodstream, and then invade normal tissues elsewhere in the body. Depending on whether or not they can spread by invasion and metastasis, tumors are classified as being either benign or malignant: benign tumors are tumors that cannot spread by invasion or metastasis, i.e., they only grow locally; whereas malignant tumors are tumors that are capable of spreading by invasion and metastasis. Biological processes known to be related to cancer include angiogenesis, immune cell infiltration, cell migration and metastasis. Cancers usually share some of the following characteristics: sustaining proliferative signalling, evading growth suppressors, resisting cell death, enabling replicative immortality, inducing angiogenesis, and activating invasion and eventually metastasis. Cancers invade nearby parts of the body and may also spread to more distant parts of the body through the lymphatic system or bloodstream. Cancers are classified by the type of cell that the tumor cells resemble, which is therefore presumed to be the origin of the tumor. In particular, the term “colon cancer” or “colon tumor” or “colon carcinoma” refers to any malignant proliferative colon cell disorder. The term “pancreatic cancer” refers to any malignant proliferative cell disorder that originates in pancreas.

Magnetic Resonance Imaging Activatable Contrast Agent of the Invention

The inventor of the present invention has developed an activatable contrast agent for magnetic resonance imaging (MRI) comprising a paramagnetic agent and a superparamagnetic agent in such physical proximity that a magnetic quenching exists between the two agents. Both the paramagnetic agent and the superparamagnetic agents of the activatable MRI contrast agent of the invention are operably linked to a oligonucleotide comprising nucleotides conferring resistance to mammalian endonucleases as well as a nucleotide sequence conferring sensitivity to a microbial nuclease but not to a mammalian endonuclease. When the MRI activatable contrast agent of the invention is in contact with a nuclease, such as a microbial nuclease, the nuclease cleaves the oligonucleotide comprised by the contrast agent of the invention at the region comprising nucleotides conferring sensibility to nuclease digestion, the paramagnetic agent and the superparamagnetic agent become separated, quenching between the two agents is ceased, and a magnetic signal derived from the activated contrast agent is detected by MRI.

Thus, in a first aspect, the invention relates to an activatable contrast agent for magnetic resonance imaging (MRI) comprising:

(i) a superparamagnetic agent,

(ii) at least one paramagnetic agent, and

(iii) at least one DNA, RNA or DNA-RNA, single or double stranded oligonucleotide, one end of said at least one oligonucleotide operably linked to the superparamagnetic agent in (i) and the other end of said at least one oligonucleotide operably linked to the at least one paramagnetic agent in (ii), wherein said at least one oligonucleotide comprises at least one region comprising nucleotides conferring resistance to mammalian endonucleases, and at least one region comprising a nucleotide sequence conferring sensitivity to a microbial nuclease but not to mammalian endonucleases, and wherein said oligonucleotide in (iii) allows magnetic quenching between the superparamagnetic agent in (i) and the at least one paramagnetic agent in (ii).

Superparamagnetic Agent

The MRI activatable contrast agent of the invention comprises a superparamagnetic agent. Particular superparamagnetic agents according to the invention include, agents comprising iron oxide or iron platinum.

Superparamagnetic iron oxide (SPIO) agents are composed of nano-sized iron oxide crystals coated with dextran or carboxydextran. Two SPIO agents are clinically approved, namely: ferumoxides (Feridex® in the USA, Endorem® in Europe) with a particle size of 120 to 180 nm, and ferucarbotran (Resovist®) with a particle size of about 60 nm. The principal effect of the SPIO particles is on T2* relaxation and thus MR imaging is usually performed using T2/T2*-weighted sequences in which the tissue signal loss is due to the susceptibility effects of the iron oxide core.

In a particular embodiment, the superparamagnetic agent of the contrast agent of the invention comprises an iron oxide nanoparticle (ION). Superparamagnetic iron oxide nanoparticles are also known as SPIONs. Commercially available SPIONs include, without limitation, Feridex® and Resovist®.

Chemical methods used to synthesize SPIONs are known by the skilled person and include, without limitation, coprecipitation, thermal decomposition, pyrolysis method, hydrothermal reactions, and sol-gel syntheses. In a particular embodiment, the iron oxide core of the nanoparticle is coated with a layer, wherein the nature of the surface coating and modification methods determine the physical and biologic properties of the SPION such as the overall size, surface charge, coating density, toxicity and degradability.

In a particular embodiment, the superparamagnetic agent of the contrast agent of the invention comprises an iron platinum particle (SIPP). Structural and magnetic characterization of said particles has been described by Taylor (Taylor R M et al. 2012 J Vac Sci Technol B Nanotechnol Microelectron 30(2): 02C101-02C101-6).

In a preferred embodiment, the superparamagnetic agent of the activatable contrast agent of the invention comprises iron oxide. In a particularly preferred embodiment, the superparamagnetic agent of the activatable contrast agent of the invention is an iron oxide nanoparticle (ION).

Paramagnetic Agent

The MRI activatable contrast agent of the invention further comprises at least one unit of a paramagnetic agent. In a particular embodiment, the MRI activatable contrast agent of the invention comprises one unit of a paramagnetic agent. In a particular preferred embodiment, the MRI activatable contrast agent of the invention comprises two or more units of a paramagnetic agent. Said two or more units of the paramagnetic agent may be the same or different paramagnetic agents. In a particular preferred embodiment, the MRI activatable contrast agent comprises at least two units of the same paramagnetic agent.

Particular paramagnetic agents according to the invention are selected from the group consisting of gadolinium-based agents, and manganese-based agents. In a particular embodiment, the paramagnetic agent is a gadolinium-based agent. Gadolinium-based agents according to the invention include, without limitation, gadolinium-DTPA (a complex of gadolinium with a chelating agent, diethylenetriaminepenta-acetic acid or DTPA; also known as magnevist), gadolinium-DOTA (a complex of gadolinium and tetraazacyclododecanetetraacetic acid), gadolinium-NOTA (a complex of gadolinium and 1, 4,7-triazacyclononane-N, N′, N″-triacetate), and gadolinium-DOTRA (a complex of gadolinium and 1, 5, 9-triazacyclododecane-N, N′, N″-triacetate). In a particular embodiment, the gadolinium-based agent is gadolinium-DTPA. Alternatively, the paramagnetic agent is a manganese-based agent. In a particular embodiment, the manganese-based agent is manganese-DPDP (manganeseII-N,N′-dipyridoxyl-ethylene-diamine-N,N′-diacetate-5,5′-bisphosphate, manganese dipyridocaldiphosphate, also known as mangafodipir).

In a preferred embodiment, the paramagnetic agent of the activatable contrast agent of the invention comprises a gadolinium-based agent. In a particularly preferred embodiment, the paramagnetic agent of the activatable contrast agent of the invention is gadolinium-DTPA.

Oligonucleotide

The MRI activatable contrast agent of the invention further comprises at least one DNA or RNA, single or double stranded oligonucleotide, one end of said at least one oligonucleotide operably linked to the superparamagnetic agent in (i) and the other end of said at least one oligonucleotide operably linked to the at least one paramagnetic agent in (ii). In a particular embodiment, the contrast agent of the invention comprises one oligonucleotide, wherein one end of the oligonucleotide (either the 5′ or 3′ end, indistinctly) is operatively linked to the superparamagnetic agent and the other end of the oligonucleotide is operably linked to the paramagnetic agent. In a particular preferred embodiment, the contrast agent comprises at least two units of the oligonucleotide, wherein one end of the oligonucleotides (either the 5′ or 3′ end, indistinctly, but preferably the same end for said at least two oligonucleotides) is operatively linked to the superparamagnetic agent, and the other end of the oligonucleotides is operably linked to the at least one paramagnetic agent.

The oligonucleotide of the contrast agent of the invention can be a DNA oligonucleotide, wherein said DNA oligonucleotide comprises deoxyribonucleotides, a RNA oligonucleotide, wherein said RNA oligonucleotide comprises ribonucleotides, or a DNA-RNA oligonucleotide, wherein said DNA-RNA oligonucleotide comprises both deoxyribonucleotides and ribonucleotides, in any proportion. In a particular embodiment, the oligonucleotide is a DNA-RNA oligonucleotide.

The oligonucleotide of the contrast agent of the invention can be a single stranded or a double stranded oligonucleotide. A single stranded oligonucleotide relates to a nucleic acid molecule that exists primarily as a single strand of nucleic acid in contrast to a double-stranded product which exists as two strands of nucleic acid which are held together by inter-strand base pairing interactions. A double stranded oligonucleotide exists in hydrogen bonded, substantially complementary, helical arrangement. In a particular embodiment, the oligonucleotide is a single stranded oligonucleotide.

The oligonucleotide of the contrast agent of the invention comprises the following elements:
at least one region comprising nucleotides conferring resistance to mammalian endonucleases, and
at least one region comprising a nucleotide sequence conferring sensitivity to a microbial nuclease but not to mammalian endonucleases.

In a particular embodiment, the nucleotides conferring resistance to mammalian endonucleases comprised by the oligonucleotide of the contrast agent of the invention are chemically modified nucleotides. Particular chemically modified nucleotides according to the invention include, without limitation, 2′-O-methyl-nucleotide, 2′-fluoro-nucleotide, a locked nucleic acid (LNA), an unlocked nucleic acid (UNA), and 2′-deoxy-2′-fluoro-β-d-arabinonucleic acid (FANA). In a particular embodiment, the oligonucleotide of the contrast agent of the invention comprises at least one chemically modified nucleotide. In a more particular embodiment, the oligonucleotide of the contrast agent of the invention comprises between 1 and 50 chemically modified nucleotides, more particularly between 2 and 20 chemically modified nucleotides. However, according to the invention, the region of the oligonucleotide comprising a nucleotide sequence conferring sensitivity to a microbial nuclease but not to mammalian nucleases may also comprise chemically modified oligonucleotides.

In a particular embodiment, the 2′-modification of the ribose of at least one position of the oligonucleotide confers resistance to degradation by mammalian endonucleases. A 2′-O-methyl-nucleotide is the result of 2′-O-methylation, a common nucleoside modification of RNA, where a methyl group is added to the 2′ hydroxyl group of the ribose moiety of a nucleoside. In a particular embodiment, the 2′-O-methyl-nucleotide is a 2′-O-methyl-purine or a 2′-O-methyl-pyrimidine. In a particular preferred embodiment, the 2′-O-methyl-nucleotide selected from the group consisting of 2′-O-methyl-citosine, 2′-O-methyl-guanine and 2′-O-methyl-uracil. However, 2′O-Me oligonucleotides remain sensitive to exonuclease degradation and must be protected at their ends with additional chemical modification(s), such as phosphorothioate (PS) linkages or addition of long terminal hairpin structures.

In a particular embodiment, the presence of at least a 2′-fluoro-nucleotide in the oligonucleotide confers resistance to degradation by mammalian endonucleases. The synthesis of 2′-fluoro-nucleotides is known by the skilled person and described by Schultz & Gryaznov (Schultz R G & Gryaznov S M 1996 Nucleic Acids Res 24(15): 2966-2973).

In a particular embodiment, the presence of at least a LNA in the oligonucleotide confers resistance to degradation by mammalian endonucleases. The term “Locked Nucleic Acid, or “LNA”, as used herein, often referred to as inaccessible RNA, relates to a modified RNA nucleotide. The ribose moiety of an LNA nucleotide is modified with an extra bridge connecting the 2′ and 4′ carbons (02′,C4′-methylene bridge). The bridge “locks” the ribose in the 3′-endo structural conformation, which is often found in the A-form of DNA or RNA. LNA nucleotides can be mixed with DNA or RNA bases in the nucleic acid whenever desired. Such oligomers are commercially available. The locked ribose conformation enhances base stacking and backbone pre-organization. This significantly increases the thermal stability (melting temperature) and hybridization affinity of LNA-modified nucleic acids, besides having improved mismatch discrimination abilities.

In a particular embodiment, the presence of at least a UNA in the oligonucleotide confers resistance to degradation by mammalian endonucleases. The term “unlocked nucleic acid” or “UNA”, as used herein, also known as inaccessible RNA, relates to a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides.

In a particular embodiment, the presence of at least a FANA in the oligonucleotide confers resistance to degradation by mammalian endonucleases. The FANA has the following structure:

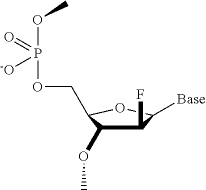

In a particular preferred embodiment of the invention, the region comprising nucleotides conferring resistance to mammalian endonucleases of the oligonucleotide comprises at least one chemically modified nucleotide, wherein said nucleotide is a 2′-O-methyl-nucleotide. Particularly preferred 2′-O-methyl-nucleotide are 2′-O-methyl-citosine, 2′-O-methyl-guanine and 2′-O-methyl-uracil.

In a particular embodiment, the nucleotide sequences conferring sensitivity to a microbial nuclease but not to mammalian endonucleases comprised by the oligonucleotide of the contrast agent of the invention are selected from the group consisting of TT, AA, TA, AT. In a preferred embodiment, said nucleotide sequence conferring sensitivity to a microbial nuclease but not to mammalian endonucleases is TT or TA. In a more preferred embodiment, said nucleotide sequence conferring sensitivity to a microbial nuclease but not to mammalian endonucleases is TT. The nucleotide sequence TT is recognized and cleaved, in a non-limiting example, by the micrococcal nuclease (MN) of S. aureus.

The at least one oligonucleotide comprised by the contrast agent of the invention allows magnetic quenching between the superparamagnetic agent and the at least one paramagnetic agent. In a particular embodiment, magnetic quenching of the paramagnetic and superparamagnetic agents is allowed by the length of the oligonucleotide, wherein said length is of at least two nucleotides, or by the presence of a nucleotide sequence in said oligonucleotide having a secondary structure. The presence of a secondary structure in the oligonucleotide of the agent of the invention involves that at least two nucleotides are complementary and interact between them by hydrogen bonds. Thus, in a particular embodiment, the presence of a secondary structure in the oligonucleotide of the agent of the invention involves auto-complementarity of the nucleotide sequence. Methods and programs for the prediction of the presence of a secondary structure in a nucleotide sequence based on sequence alignment are known by the skilled person and include, without limitation, the software for performing BLAST analyses, which is publicly available through the National Center for Biotechnology Information (NCBI) website, the RNAstructure software (Reuter J S & Mathews D H 2010 BMC Bioinformatics 11: 129), and mfold (Zuker M 2003 Nucleic Acids Res 31(13): 3406-3415).

Particular preferred sequences of the oligonucleotide comprised by the contrast agent of the invention include the following nucleotide sequences:

(SEQ ID NO: 1)
5′-mCmUmCmGTTmCmGmUmUmC-3′,
(SEQ ID NO: 2)
5′-TTmCmGmCmUmUmCmGmGmCmGmAmA-3′,
(SEQ ID NO: 3)
5′-mCTAmCmGmCmUmUmCmGmGmCmGTAmG-3′,
(SEQ ID NO: 4)
5′-mCmUmCmGTTmCmGmUmUmC-3′
(SEQ ID NO: 5)
5′-mCmUmC mG TT mCmGmU mUmC
(five repetition of mU)-3′,
(SEQ ID NO: 6)
5′-mCmUmC mG TT mCmGmU mUmC
(25 repetitions of mU)-3′,
(SEQ ID NO: 7)
5′-mCmUmC mG TT mCmGmU mUmC
(50 repetitions of mU)-3′,
(SEQ ID NO: 8)
5′-mCmUmC mG mGmG mCmGmU mUmC-3′,
(SEQ ID NO: 9)
5′-TTmCmGmCmUmUmCmGmGmCmGmAmA-3′,
(SEQ ID NO: 10)
5′-mUmCmUmCmCmUfAfAfAmUmCmCmUmCmU-3′
and
(SEQ ID NO: 11)
5′-fUfCfUfCfGfUfAfCfGfUtUfC-3′.

wherein m represents a 2′O-methyl nucleotide, in particular, mC: 2′O-methyl cytidine, mU: 2′O-methyl uridine, mG: 2′O-methyl guanosine, mA: 2′O-methyl adenosine. The sequence that confers sensitivity to a microbial nuclease but not to mammalian endonucleases is shown in bold.

However, it should be noted that the specific sequence of the oligonucleotide is not critical, since certain combinations of purines and pyrimidines are susceptible to bacterial endonucleases, while resisting mammalian nucleases. Additionally, some endonucleases cleave single-stranded nucleic acid molecules, while others cleave double-stranded nucleic acid molecules.

The oligonucleotides of the invention are synthesized using conventional phosphodiester linked nucleotides and using standard solid or solution phase synthesis techniques which are known in the art.

The oligonucleotides are short, such as between 2-65 nucleotides in length (or any value in between). In certain embodiments, that oligonucleotide is between 10-20 nucleotides in length. In certain embodiments, that oligonucleotide is between 11-16 nucleotides in length. In general, shorter sequences will give better signal to noise ratios than longer probes and will therefore be more sensitive. However, in certain embodiments, shorter probes might not be the best substrate for the nuclease, so some degree of empiric optimization for length is needed. In certain embodiments, the oligonucleotide comprises 0-50% purines (or any value in between). In certain embodiments the oligonucleotide comprises 100% pyrimidines.

In a particular embodiment, the length of the oligonucleotide is such that the distance between the paramagnetic agent, preferably gadolinium, and the superparamagnetic agent, preferably an iron oxide nanoparticle, is between 5 and 100 nm, more particularly between 5 and 75 nm, even more particularly between 5 and 50 nm, yet more particularly between 5 and 25 nm. The distance between the paramagnetic agent, preferably gadolinium, and the superparamagnetic agent, preferably an iron oxide nanoparticle, can be measured by any suitable technique known by the skilled person. For example, transmission electron microscopy (TEM) can be used to determine the size of the superparamagnetic core (for example, the size of the iron oxide nanoparticle) of the contrast agent and photon correlation spectroscopy (PCS) can be used to provide the total size of the contrast agent, including the superparamagnetic agent, the nucleic acid and the paramagnetic agent, as described by Bordelon et al, Journal of Applied Physics 2011, 109: 124904-1 and Shin et al, ACs Nano 2014, 8(4): 3393-3401. The distance between the superparamagnetic agent and the paramagnetic agent can be calculated based on the difference between the size of the contrast agent and the size of the superparamagnetic agent, assuming that the superparamagnetic agent is located in the center of the contrast agent.

In a particular embodiment of the invention, the paramagnetic agent and/or the superparamagnetic agent of the activatable contrast agent of the invention are operably linked to the oligonucleotide by means of a linker.

In a particular embodiment, an aliphatic or ethylene glycol linker (as are well known to those with skill in the art) is used. In a particular embodiment, the linker is a phosphodiester linkage. In a particular embodiment, the linker is a phosphorothioate linkage. In a particular embodiment, other modified linkages are used in order to make these linkages more stable, thereby limiting degradation to the nucleases.

In a particular embodiment, the linker is a binding pair. The term “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-hormone receptor, receptor-ligand, enzyme-substrate, IgG-protein A, antigen-antibody, avidin/biotinylated molecules, enzyme/enzyme inhibitor, protein A/antibody, protein G/antibody, and the like. In certain embodiments, a first member of the binding pair comprises avidin or streptavidin and a second member of the binding pair comprises biotin. In certain embodiments, the oligonucleotide is linked to the paramagnetic agent and/or to the superparamagnetic agent by means of a covalent bond.

In another particular embodiment, the oligonucleotide is operatively linked to the paramagnetic agent, preferably gadolinium, by a chelating agent. The term “chelating agent” as used herein, is an agent that binds to a metal ion such as gadolinium (Gd³⁺), forming a metal complex known as a chelate. In some embodiments, a chelating agent is a ligand. In some embodiments, a chelating agent is an atom. In some embodiments, a chelating agent is an ion. In some embodiments, a chelating agent enhances the binding of the oligonucleotide to the paramagnetic agent, preferably gadolinium. In a particular embodiment, the chelating agent is diethylenetriaminepentaacetic acid (DTPA). In a more particular embodiment, the oligonucleotide is operatively linked to the paramagnetic agent, preferably gadolinium, by the modification of one of its ends, preferably the 5′ end, with a chelating agent, preferably DTPA.

In another particular embodiment, the oligonucleotide is operatively linked to the superparamagnetic agent, preferably an iron oxide nanoparticle, by a crosslinker. The term “crosslinker”, as used herein, refers to multi-Functional monomers capable of forming two or more covalent bonds between polymer molecules or particles of the same or different type. When the olignonucleotide is operatively linked to the superparamagnetic agent by a crosslinker, the olignonucleotide may be chemically modified in order to allow the reaction with the crosslinker. In a particular embodiment the crosslinker is a sulfhydryl-reactive group coupled to the superparamagnetic agent, preferably the iron oxide nanoparticle, and the oligonucleotide is modified with a sulfhydryl group in its 5′ or 3′ end. Examples of sulfhydryl-reactive groups include haloacetyls, maleimides, aziridines, acryloyls, arylating agents, vinylsulfones, pyridyl disulfides, TNB-thiols and disulfide reducing agents. Most of these groups conjugate to sulfhydryls by either alkylation (usually the formation of a thioether bond) or disulfide exchange (formation of a disulfide bond). In a particular embodiment, the sulfhydryl-reactive group is maleimide. In a more particular embodiment, one of the ends of the oligonucleotide, preferably the 3′ end, modified with a sulfhydryl group reacts with the maleimide coupled to the iron oxide nanoparticle forming a thioether bond, therefore allowing the conjugation of the oligonucleotide and the iron oxide nanoparticle.

When the oligonucleotide is operatively linked to the superparamagnetic agent by a crosslinker, the superparamagnetic agent may be modified with a reactive group. When the crosslinker is a sulfhydryl-reactive group, for example maleimide, the superparamagnetic agent, preferably the iron oxide nanoparticle, may be covered with a matrix to functionalize the superparamagnetic agent with the sulfhydryl-reactive group. Said matrix can be any matrix which can be chemically linked to the sulfhydryl-reactive group, in particular maleimide. In a particular embodiment, the matrix is a polymeric matrix, for example a dextran matrix.

Chemistries that can be used to link the paramagnetic agent and the superparamagnetic agent to the oligonucleotide are known in the art, including without limitation disulfide linkages, amino linkages, and covalent linkages. In certain embodiments, aliphatic or ethylene glycol linkers can be used.

In an even more particular embodiment, the oligonucleotide is operatively linked to the paramagnetic agent, preferably gadolinium, by the modification of one of its 5′ end with a chelating agent, preferably DTPA and to the superparamagnetic agent, preferably the iron oxide nanoparticle, by a thioether bond between a sulfhydryl group in its 3′ end and a maleimide coupled to the superparamagnetic agent, preferably the iron oxide nanoparticle.

The present invention relates as well to a kit comprising an activatable contrast agent for MRI reagent according to the invention and additional reagents suitable for detecting microbial nuclease activity. Thus, the kit may optionally comprise at least one of the following: instructions for use, a positive control nuclease (e.g., ribonuclease), RNase-free water, and a buffer. Said instructions can be found in the form of printed material or in the form of an electronic support which can store instructions such that they can be read by a subject, such as electronic storage media (magnetic disks, tapes and the like), optical media (CD-ROM, DVD) and the like. The media can additionally or alternatively contain internet websites providing said instructions. It is also provided that the kits may include RNase-free laboratory plasticware, for example, thin-walled, UV transparent microtubes and/or multiwell plates for a high-throughput format.

In a particular embodiment, the superparamagnetic agent, for example the iron oxide nanoparticle, and the paramagnetic agent, for example gadolinium, are separated by a polymeric matrix. In a more particular embodiment, the polymeric matrix is a dextran matrix.

In a particular embodiment, the activatable contrast agent of the invention is selected from:

Gadolinium-DTPA-SEQ ID NO: 4-ION,

Gadolinium-DTP -SEQ ID NO: 5-ION,

Gadolinium-DTPA-SEQ ID NO: 7-ION,

Gadolinium-DTPA-SEQ ID NO: 8-ION,

Gadolinium-DTPA-SEQ ID NO: 9-ION,

Gadolinium-DTPA-SEQ ID NO: 10-ION and

Gadolinium-DTPA-SEQ ID NO: 11-ION,

wherein the iron oxide nanoparticle (ION) is coated by a dextran matrix modified with maleimide, and wherein the oligonucleotide of SEQ ID 4 to 11 is operatively linked to the iron oxide nanoparticle (ION) by a thioether bond between its SH-modified 3′ end and the maleimide linked to the dextran matrix.

### Pharmaceutical Composition of the Invention

The present invention also relates to a pharmaceutical composition comprising the activatable contrast agent for MRI as described above.

The pharmaceutical compositions containing the agent according to the invention can occur at any pharmaceutical form of administration considered appropriate for the selected administration route, for example, by systemic, oral, parenteral or topical administration, for which it will include the pharmaceutically acceptable excipients necessary for formulation of the desired method of administration. Thus, one or more suitable unit dosage forms of the activatable contrast agent of the invention can be administered by a variety of routes including parenteral, including by intravenous and intramuscular routes, as well as by direct injection into a particular tissue.

The effective quantity of the activatable contrast agent of the invention can vary within a wide range and, in general, will vary depending on the particular circumstances of application, duration of the exposure and other considerations.The amount administered will vary depending on various factors including, but not limited to, the composition chosen, the particular disease, the weight, the physical condition, and the age of the subject. Such factors can be readily determined by the clinician employing animal models or other test systems, which are well known to the art.

When the activatable contrast agent of the invention is prepared for administration, in certain embodiments it is combined with a pharmaceutically acceptable carrier, diluent or excipient to form a pharmaceutical formulation, or unit dosage form. The total active ingredient (i.e., activatable contrast agent) in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the activatable contrast agent of the invention can be prepared by procedures known in the art using well known and readily available ingredients.

Solid dosage forms for oral administration may include conventional capsules, sustained release capsules, conventional tablets, sustained-release tablets, chewable tablets, sublingual tablets, effervescent tablets, pills, suspensions, powders, granules and gels. At these solid dosage forms, the active compounds can be mixed with at least one inert excipient such as sucrose, lactose or starch. Such dosage forms can also comprise, as in normal practice, additional substances other than inert diluents, e.g. lubricating agents such as magnesium stearate. In the case of capsules, tablets, effervescent tablets and pills, the dosage forms may also comprise buffering agents. Tablets and pills can be prepared with enteric coatings.

Liquid dosage forms for oral administration may include emulsions, solutions, suspensions, syrups and elixirs pharmaceutically acceptable containing inert diluents commonly used in the technique, such as water. Those compositions may also comprise adjuvants such as wetting agents, emulsifying and suspending agents, and sweetening agents, flavoring and perfuming agents.

The activatable contrast agent of the invention may be formulated for parenteral administration (e.g., by injection, for example, bolus injection or continuous infusion) and may be presented in unit dose form in ampules, pre-filled syringes, small volume infusion containers or in multi-dose containers with an added preservative. The probe may take such forms as suspensions, solutions, or emulsions in oily or aqueous vehicles, and may contain formulatory agents such as suspending, stabilizing and/or dispersing agents. Alternatively, the probe may be in powder form, obtained by aseptic isolation of sterile solid or by lyophilization from solution, for constitution with a suitable vehicle, e.g., sterile, pyrogen-free water, before use. Injectable preparations, for example, aqueous or oleaginous suspensions, sterile injectable may be formulated according with the technique known using suitable dispersing agents, wetting agents and/or suspending agents. Among the acceptable vehicles and solvents that can be used are water, Ringer's solution and isotonic sodium chloride solution. Sterile oils are also conventionally used as solvents or suspending media.

The composition comprising the activatable contrast agent of the invention can additionally include conventional excipients, e.g. pharmaceutically acceptable carriers suitable for parenteral application which do not react damaging with the active compounds. Suitable pharmaceutically acceptable vehicles include, for example, water, salt solutions, alcohol, vegetable oils, polyethylene glycols, gelatin, lactose, amylose, magnesium stearate, talc, surfactants, silicic acid, viscous paraffin, perfume oil, monoglycerides and diglycerides of fatty acids, fatty acid esters petroetrals, hydroxymethyl cellulose, polyvinylpyrrolidone and similars. Optional additional ingredients of the composition include diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0, saline solutions, and water.

Several drug delivery systems are known and can be used to administer the agent or composition of the invention, including, for example, encapsulation in liposomes, microbubbles, emulsions, microparticles, microcapsules and similars. The required dosage can be administered as a single unit or in a sustained release form.

Sustainable-release forms and appropriate materials and methods for their preparation are described in, for example, “Modified-Release Drug Delivery Technology”, Rathbone, M. J. Hadgraft, J. and Roberts, M. S. (eds.), Marcel Dekker, Inc., New York (2002), “Handbook of Pharmaceutical Controlled Release Technology”, Wise, D. L. (ed.), Marcel Dekker, Inc. New York, (2000).

Even though individual needs vary, determination of optimal ranges for effective amounts of the agent of the invention belongs to the common experience of those experts in the art. In general, the dosage needed to provide an effective amount of such compound, which can be adjusted by one expert in the art will vary depending on age, health, fitness, sex, diet, weight, degree of alteration, frequency of treatment and the nature and extent of impairment or illness, medical condition of the patient, route of administration, pharmacological considerations such as activity, efficacy, pharmacokinetic and toxicology profile of the particular compound used, if using a system drug delivery, and if the compound is administered as part of a combination of drugs.

### Diagnostic Method of the Invention

As previously described, the MRI activatable contrast agent of the invention becomes activated in the presence of nuclease activity. In particular, S. aureus is one of the most common microbial agents responsible for focal infections in people. S. aureus secretes a nuclease known as micrococcal nuclease (MN), that exhibits robust DNase and RNase activities, and that is active on both single-and double-stranded substrates. Bacterial nuclease activity has been characterized as well in Streptococcus pneumoniae (endA nuclease) and Mycobacterium tuberculosis (endonuclease IV). Nuclease activity is associated also to virus, including without limitation cytomegalovirus (UL98 endonuclease) and to fungi, including without limitation Aspergillus (nuclease S1).

On the other hand, overexpression of proteins containing domains similar to those of the microbial nucleases has been described in tumor cells. In particular, increased expression of SND1 (Staphylococcal nuclease homology domain containing 1) protein has been closely related to carcinogenesis, progression and prognosis of colon cancer (see Wang L et al. 2012 Mol Biol Rep 39: 10497-10504). Therefore, detection of nuclease activity may also be associated to the presence of tumor cells, such as colon tumor cells (Tsuchiya N et al. 2007 Cancer Res 67 19: 9568-9576) or pancreatic tumor cells (Fernandez E et al. 2000 Eur J Biochem 267: 1484-1494, Peracaula R et al. 2003 Glycobiology 13:227-244).

Thus, the present invention relates to a magnetic resonance imaging (MRI) method for detecting a nuclease activity in a subject, wherein said nuclease activity is a microbial nuclease activity or a tumor cell nuclease activity, which comprises:
(i) administering a contrast agent according to the invention or a pharmaceutical composition thereof according to the invention to said subject, and
(ii) detecting activated contrast agent by MRI.

Thus, the MRI method of the invention for detection of nuclease activity in a subject involves the administration to a subject of the MRI activatable contrast agent according to the invention, or the administration of a pharmaceutical composition comprising said contrast agent. Suitable administration routes of the agent of the invention or pharmaceutical composition thereof have been previously described in the context of the pharmaceutical composition of the invention and include, without limitation, systemic, oral, and parenteral administration. In a particular embodiment, the administration is parenteral, including intravenous and intramuscular routes, or by direct injection into a particular tissue.

The contrast agent or the pharmaceutical composition according to the invention is administered to a subject. In a particular embodiment, the subject is suspected or candidate to suffer from a microbial infection, particularly from a bacterial infection, a fungal infection or a viral infection. In a particular alternative embodiment, the subject is suspected or candidate to suffer from cancer, particularly from colon cancer or pancreatic cancer.

In a particular embodiment, the subject is suspected or candidate to suffer from a bacterial infection. Species of a bacterial genus according to the invention include, without limitation, Acinetobacter, Aerococcus, Bacillus, Bacteroides, Bordetella, Campylobacter, Clostridium, Corynebacterium, Chlamydia, Citrobacter, Enterobacter, Enterococcus, Escherichia, Francisella, Helicobacter, Haemophilus, Klebsiella, Legionella, Listeria, Mycobacterium, Micrococcus, Mobilincus, Moraxella, Mycobacterium, Mycoplasma, Neisseria, Oligella, Pasteurella, Prevotella, Porphyromonas, Pseudomonas, Propionibacterium, Proteus, Salmonella, Serratia, Shigella, Staphylococcus, Streptococcus, Treponema, Vibrio, and Yersinia. Bacteria causing infection in a subject according to the present invention include, without limitation, Acinetobacter calcoaceticus, Aerococcus viridans, Bacillus anthracis, Bacillus cereus, Bacteroides fragilis, Bordetella pertussis, Bordetella parapertussis, Campylobacter jejuni, Clostridium difficile, Clostridium perfringens, Corynebacterium sp., Chlamydia pneumoniae, Chlamydia trachomatis, Citrobacter freundii, Enterobacter aerogenes, Enterococcus gallinarum, Enterococcus faecium, Enterobacter faecalis (e.g., ATCC 29212), Escherichia coli (e.g., ATCC 25927), Francisella philomiragia (GA01-2810), Francisella tularensis (LVSB), Gardnerella vaginalis, Helicobacter pylori, Haemophilus influenzae (e.g., ATCC 49247), Helicobacter pilori, Klebsiella pneumoniae, Legionella pneumophila (e.g.,ATCC 33495), Listeria monocytogenes (e.g., ATCC 7648), Micrococcus sp. strain ATCC14396, Moraxella catarrhalis, Mycobacterium kansasii, Mycobacterium gordonae, Mycobacterium fortuitum, Mycobacterium tuberculosis, Mycoplasma pneumoniae, Mycoplasma hominis, Neisseria meningitis (e.g., ATCC 6250), Neisseria gonorrhoeae, Oligella urethralis, Pasteurella multocida, Pseudomonas aeruginosa (e.g., ATCC 10145), Propionibacterium acnes, Proteus mirabilis, Proteus vulgaris, Salmonella sp. strain ATCC 31194, Salmonella enterica, Salmonella typhimurium, Serratia marcescens (e.g., ATCC 8101), Staphylococcus aureus (e.g., ATCC25923), Staphylococcus epidermidis (e.g., ATCC 12228), Staphylococcus lugdunensis, Staphylococcus saprophyticus, Streptococcus pneumoniae (e.g., ATCC 49619), Streptococcus pyogenes, Streptococcus agalactiae (e.g., ATCC 13813), Treponema pallidum, Vibrio cholerae, Viridans group streptococci (e.g., ATCC 10556), Yersinia pseudotuberculosis (PB 11+), Yersinia enterocolitica, 0:9 serotype, and Yersinia pestis (P14−). In a particular embodiment, the subject is suspected or candidate to suffer from a bacterial infection caused by a bacteria selected from the group consisting of Campylobacter jejuni, Clostridium tetani, Enterobacter spp, Enterococcus faecalis, Enterococcus faecium, Escherichia coli, Haemophilus influenzae, Helicobacter pylori, Legionella, Mycobacterium tuberculosis, Neisseria, Pseudomonas aeruginosa, Salmonella enterica, Shigella, Staphylococcus aureus, Staphylococcus epidermidis, Streptococcus pneumoniae, Treponema pallida, Vibrio cholerae and Yersinia pestis. In a more particular embodiment, the subject is suspected or candidate to suffer from a bacterial infection caused by Staphylococcus aureus, Streptococcus pneumonia, or Mycobacterium tuberculosis. Staphylococcus aureus is a Gram-positive coccal bacterium and common causative agent of skin infections (including pimples, impetigo, furuncles, carbuncles), respiratory diseases (pneumonia) and food poisoning. Streptococcus pneumoniae is a Gram-positive bacteria that may become pathogenic in susceptible individuals, such as elderly and immunocompromised people and children, causing community acquired pneumonia and meningitis in children and the elderly, or septicemia in HIV-infected persons. Mycobacterium tuberculosis is a pathogenic bacterial species and causative agent of most cases of tuberculosis.

In a particular embodiment, the subject is suspected or candidate to suffer from a fungal infection. Fungus causing infections in a subject according to the present invention include, without limitation, Aspergillus, Candida, and Pneumocystis jirovecii. In a particular embodiment, the subject is suspected or candidate to suffer from a fungal infection caused by Aspergillus. Aspergillus is a member of the deuteromycetes fungi causing a group of diseases known as aspergillosis, including allergic bronchopulmonary aspergillosis, acute invasive aspergillosis, disseminated invasive aspergillosis, and aspergilloma.

In a particular embodiment, the subject is suspected or candidate to suffer from a viral infection. Virus according to the invention include, without limitation, human immunodeficiency virus (HIV), influenza virus, dengue virus, herpes simplex virus, varicella-zoster virus, Epstein-barr virus, cytomegalovirus, papillomavirus, BK virus, JC virus, smallpox, hepatitis B virus, parvovirus, astrovirus, Norwalk virus, coxsackie virus, hepatitis A virus, poliovirus, rhinovirus, severe acute respiratory syndrome virus, hepatitis C virus, yellow fever virus, West Nile virus, rubella virus, hepatitis E virus, Lassa virus, haemorrhagic fever virus, Ebola virus, Marburg virus, measles virus, mumps virus, parainfluenza virus, respiratory syncytial virus, metapneumovirus, Hendra virus, Nipah virus, rabies virus, hepatitis D virus, rotavirus, orbivirus, coltivirus or Banna virus. In a particular embodiment, virus causing infections in a subject according to the present invention are selected from the group consisting of cytomegalovirus, Epstein-Barr virus, hepatitis virus and herpervirus. In a particular embodiment, the subject is suspected or candidate to suffer from a viral infection caused by a cytomegalovirus. The cytomegalovirus (CMV) is a member of the Herpesviridae family of virus, this family including members such as herpes simplex virus, Epstein-Barr virus, or varicella zoster virus.

In a particular alternative embodiment, the subject is suspected or candidate to suffer from cancer. Cancer causing disease in a subject according to the present invention includes any type of cancer where a nuclease activity can be measured as a specific biomarker including, without limitation, colon cancer and pancreatic cancer.

In a particular embodiment, the tumor cell nuclease activity is a SND1 nuclease activity. The term “SND1 nuclease activity”, as used herein, refers to the nuclease activity of the SND1 (Staphylococcal nuclease homology domain containing 1) protein. In humans SND1 corresponds to the protein identified with accession No.Q7KZF4 (version 127 of the entry and version 1 of the sequence as of 16 Sep. 2015 of UniProt) and secondary accession No. Q13122, Q96AG0.

Detection of the activatable contrast agent of the invention is performed by MRI. The contrast agent of the invention is designed so that the longitudinal spin-lattice magnetic relaxation (T1) of the paramagnetic agent is quenched by the magnetic field provided by the proximity to the superparamagnetic agent. However, upon cleavage of the oligonucleotide of the contrast agent by a nuclease, particularly a microbial nuclease or a tumor cell nuclease, the paramagnetic agent diffuses away from the superparamagnetic agent, allowing the recovery of its magnetic relaxation properties (unquenching), which can be measured by MRI technology.

Accordingly, different parameters associated to the activatable contrast agent can be measured in order to determine if the contrast agent becomes or not activated, i.e. if nuclease activity exists. Parameters that can be measured in order to determine activation of the contrast agent of the invention include magnetic signal intensity (brightness of the image) and T1 time.

Signal intensity is determined by factors such as the radiofrequency pulse and gradient waveforms used to obtain the image, intrinsic T1 and T2 tissue characteristics, and tissue proton density. By controlling the radio frequency pulse and gradient waveforms, computer programs produce specific pulse sequences that determine how an image is obtained (weighted) and how various tissues appear. Thus, in a particular embodiment, detection of the activated contrast agent by MRI is measured as signal intensity derived from said agent, wherein increased signal intensity relates to increased detection of the activated contrast agent.

T1 parameter, also known as longitudinal relaxation time, relates to a time constant which determines the rate at which excited protons return to equilibrium within the lattice. It involves measure of the time taken for spinning protons to re-align with the external magnetic field. The magnetization will grow after excitation from zero to a value of about 63% of its final value in a time of Tl. Thus, in an alternative particular embodiment, detection of the activated contrast agent by MRI is measured as T1 parameter, wherein a decreased T1 value relates to an increased detection of the activated contrast agent.

Thus, activation of the contrast agent according to the invention is detected by MRI. Any MRI equipment available in the art is suitable to perform the method of the invention including, without limitation, equipments from GE Healthcare, Philips, and Hitachi. This step can be done by a skilled person in the art, preferably a specialized facultative or technician, by scanning with MRI equipment.

In a particular embodiment, the method for detecting a nuclease activity in a subject further comprises comparing the signal of the activated contrast agent obtained in (ii) to a reference value, wherein if the contrast agent shows a signal higher than a reference value, then nuclease activity is detected in the subject and if the contrast agent shows a signal similar to or lower than a reference value, then nuclease activity is not detected in the subject. Suitable reference values are described below and incorporated herein.

Alternatively, the present invention relates as well to a method for the diagnosis of a microbial infection in a subject that comprises the administration of a contrast agent or a pharmaceutical composition thereof according to the invention to said subject, and detection of said contrast agent by MRI, wherein if the activated contrast agent is detected, then the subject is diagnosed with a microbial infection, and if the activated contrast agent is not detected, then the subject is not diagnosed with a microbial infection.

The invention also relates to a method for the diagnosis of a cancer in a subject, particularly colon cancer or pancreatic cancer, that comprises the administration of a contrast agent or a pharmaceutical composition thereof according to the invention to said subject and detection of said activated contrast agent by MRI, wherein if the activated contrast agent is detected, particularly in the colon or in the pancreas, then the subject is diagnosed with colon cancer or with pancreatic cancer, respectively, and if the activated contrast agent is not detected, then the subject is not diagnosed with colon cancer or with pancreatic cancer.

In a particular embodiment, the diagnosis of a microbial infection or of a cancer comprises detection of the signal derived from the contrast agent and comparison of said signal from the contrast agent to a reference value, wherein if the contrast agent shows a signal higher than a reference value, then the subject suffers from microbial infection or from cancer and if the contrast agent shows a signal similar to or lower than a reference value, then the subject does not suffer from microbial infection or from cancer. Suitable references values are described below and incorporated herein.

The term “diagnosis”, as used herein, generally relates to the process by which a disease, nosological entity, syndrome, or any disease-health condition is identified. Particularly, the term “diagnosis of a microbial infection” relates to the capacity to identify or detect the presence of an infection caused by a microorganism in a subject. Also particularly, the term “diagnosis of colon cancer” relates to the capacity to identify or detect the presence of a colon tumor in the colon of a subject. Also particularly, the term “diagnosis of pancreatic cancer” relates to the capacity to identify or detect the presence of a pancreatic tumor in the pancreas of a subject. This detection, as it is understood by a person skilled in the art, does not claim to be correct in 100% of the analyzed samples. However, it requires that a statistically significant amount of the analyzed samples are classified correctly. The amount that is statistically significant can be established by a person skilled in the art by means of using different statistical tools; illustrative, non-limiting examples of said statistical tools include determining confidence intervals, determining the p-value, the Student's t-test or Fisher's discriminant functions, etc. (see, for example, Dowdy and Wearden, Statistics for Research, John Wiley & Sons, New York 1983). The confidence intervals are preferably at least 90%, at least 95%, at least 97%, at least 98% or at least 99%. The p-value is preferably less than 0.1, less than 0.05, less than 0.01, less than 0.005 or less than 0.0001. The teachings of the present invention preferably allow correctly diagnosing in at least 60%, in at least 70%, in at least 80%, or in at least 90% of the subjects of a determined group or population analyzed.

### Microbial Infection Detection Method of the Invention

The invention relates as well to an in vitro method for determining whether a subject suffers or not from a microbial infection that comprises
(i) contacting a sample from said subject with a MRI activatable contrast agent according to the invention or with a pharmaceutical composition thereof,
(ii) detecting the signal derived from the contrast agent,and
(iii) comparing the signal of the contrast agent detected in (ii) to a reference value,
   wherein
   if the contrast agent shows a signal detectable by MRI higher than a reference value, then the subject suffers from a microbial infection, or
   if the contrast agent shows a signal detectable by MRI similar to or lower than a reference value, then the subject does not suffer from a microbial infection.

Thus, in a first step, the in vitro method of the invention for determining whether a subject suffers or not from a microbial infection comprises contacting a sample from said subject with a MRI activatable contrast agent according to the invention or with a pharmaceutical composition thereof. The sample from the subject suspected to suffer from a microbial infection and the

MRI activatable contrast agent are put into contact and incubated under conditions suitable for the interaction of the contrast agent with the sample. Particular conditions for the interaction of the contrast agent with the sample may be determined by the skilled person and include, without limitation, temperature, pH, incubation time and contrast agent concentration. In a particular embodiment, suitable conditions for the interaction of the contrast agent according to the invention and the sample include temperature 37° C., pH 7.4, detection in less than 1 hour, and a concentration able to differentiate between healthy and disease conditions) so that a signal is detected by MRI if microbial nuclease activity, and therefore microbial infection, is present in the sample.

Samples according to the invention include, without limitation, tissues or biological fluids such as blood, saliva, plasma, serum, urine, cerebrospinal liquid (CSF), feces, a surgical specimen, a specimen obtained from a biopsy, and a tissue sample embedded in paraffin. Particularly preferred samples include blood samples, urine samples and biopsy samples. Methods for isolating samples are well known to those skilled in the art. In particular, methods for obtaining a sample from a biopsy include gross apportioning of a mass, or micro-dissection or other art-known cell-separation methods. In order to simplify conservation and handling of the samples, these can be formalin-fixed and paraffin-embedded or first frozen and then embedded in a cryosolidifiable medium, such as OCT-Compound, through immersion in a highly cryogenic medium that allows rapid freeze. In a second step, the in vitro method of the invention for determining whether a subject suffers or not from a microbial infection comprises detecting the signal shown by the contrast agent of the invention. Any MRI equipment available in the art is suitable to perform the method of the invention including, without limitation, equipments from GE Healthcare, Philips, and Hitachi. In an alternative embodiment, detection of a signal from the activated contrast agent is performed by nuclear magnetic resonance (NMR). Devices for performing NMR are known in the art and include, without limitation, Bruker Minispec mg 60.

Parameters associated to the signal derived from the contrast agent have been previously described and include, without limitation, signal intensity and T1 time. In a particular embodiment, signal intensity is determined, wherein detection of the activated contrast agent by MRI is measured as signal intensity derived from said agent. In a particular alternative embodiment, T1 value is determined, wherein detection of the activated contrast agent is measured as the T1 value derived from said agent.

In a third step, the in vitro method of the invention for determining whether a subject suffers or not from a microbial infection comprises determining whether the signal detectable by MRI and due to the contrast agent of the invention in the sample from the subject is modified when compared to a reference value, wherein if the contrast agent shows a signal detectable by MRI higher than the reference value, then the subject suffers from a microbial infection, and wherein if the contrast agent shows a signal detectable by MRI similar to or lower than the reference value, then the subject does not suffer from a microbial infection.

The term “reference value”, as used herein in the context of the in vitro method for determining whether a subject suffers or not from a microbial infection, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested. In a particular embodiment, the reference value corresponds to the signal detected by MRI due to the activatable contrast agent of the invention in a control sample, wherein said control sample is a sample obtained from a healthy subject or from a subject not diagnosed with a microbial infection. In a particular alternative embodiment, the reference value corresponds to the signal detected by MRI in a control sample obtained from a healthy subject or from a subject not diagnosed with a microbial infection in the absence of the activatable contrast agent of the invention. The reference value for the signal detectable by MRI may correspond to any of the parameters associated to the activatable contrast agent that can be measured in order to determine if the contrast agent becomes or not activated. In particular, the reference value relates to parameters of a contrast agent including magnetic signal intensity and T1 time. In a more particular embodiment, the reference value relates to magnetic signal intensity or to T1 time value as determined in a control sample obtained from a healthy subject or from a subject not diagnosed with a microbial infection.

The term “high levels”, in relation to the signal as detected by MRI, relates to any level of the signal due to the activatable contrast agent of the invention detectable by MRI in a sample higher than the signal in a control sample or reference value. Thus, the levels are considered to be higher than its reference value when it is at least at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more higher than its reference value.

The term “low levels”, in relation to the signal as detected by MRI, relates to any level of the signal due to the activatable contrast agent of the invention detectable by MRI in a sample lower than the signal in a control sample or reference value. Thus, the levels are considered to be lower than its reference value when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more lower than its reference value.

The term “similar levels” or “equal levels”, in relation to the signal as detected by MRI, relates to any level of the signal due to the activatable contrast agent of the invention detectable by MRI in a sample similar to that of the signal in a control sample or reference value. Thus, the levels are considered to be similar to those levels of the reference value when they differ in less than 5%, less than 4%, less than 3%, less than 2%, less than 1%, or less than 0.5%.

In a particular embodiment, detection of the activated contrast agent by MRI is measured as signal intensity derived from said agent, wherein an increased signal intensity derived from said activated agent in comparison to the reference value is indicative of a higher signal derived from said activated agent and, therefore, of activation of the contrast agent related to nuclease activity derived from a microbial infection.

In a particular alternative embodiment, detection of the activated contrast agent is measured as the T1 value derived from said agent, wherein a decreased T1 value derived from said activated agent in comparison to the reference value is indicative of a higher signal derived from said activated agent and, therefore, of activation of the contrast agent related to nuclease activity derived from a microbial infection.

In a particular embodiment, the microbial infection to be determined by the in vitro method of the invention is caused by a microorganism selected from the group comprising bacteria, virus, and fungi. Exemplary, non-limiting bacteria, virus and fungi according to the invention have been previously mentioned in the context of the diagnostic method of the invention and incorporated herein. In a particular embodiment, bacteria causing infections in a subject that can be determined by the method of the invention are selected from the group consisting of Staphylococcus aureus (by detecting micrococcal nuclease of S. aureus), Streptococcus pneumoniae (by detection of endA nuclease of S. pneumoniae), and Mycobacterium tuberculosis (by detection of endonuclease IV of M. tuberculosis). In a particular embodiment, virus causing infections in a subject that can be determined by the method of the invention is a cytomegalovirus (by detection of UL98 endonuclease). In a particular embodiment, fungi causing infections in a subject that can be determined by the method of the invention is Aspergillus (by detection of nuclease S1).

The term “micrococcal nuclease”, abbreviated MN, also known as thermonuclease, relates to an endonuclease of Staphylococcus aureus, in particular to the nuclease identified with accession No. P00644.1, GI: 128852 in the NCBI database (231 aa, sequence version as of 1 Oct. 2014).

The term “endA nuclease”, as used herein, relates to a nuclease from Streptococcus pneumoniae, in particular to the nuclease identified with accession No. P0A3S3.1, GI:61229164 in the NCBI database (274 aa, sequence version as of 1 Oct. 2014).

The term “endonuclease IV”, as used herein, relates to a nuclease from Mycobacterium tuberculosis, in particular to the nuclease identified with accession No. KFZ75824.1, GI: 683481929 in the NCBI database (252 aa, sequence version as of 10 Sep. 2014).

The term “UL98 nuclease”, as used herein, relates to a nuclease from a cytomegalovirus, in particular to the nuclease identified with accession No. P16789.2, GI: 259016230 in the NCBI database (584 aa, sequence version as of 1 Oct. 2014).

The term “nuclease 5 1”, as used herein, relates to a nuclease from Aspergillus, particularly from Aspergillus oryzae (strain ATCC 42149/RIB 40), in particular to the nuclease identified with accession No. P24021 (287 aa, version 89 of the entry and version 2 of the sequence as of 1 Oct. 2014 of UniProt) and secondary accession No. Q00235(287 aa, version 89 of the entry and version 2 of the sequence as of 1 Oct. 2014 of UniProt).

### Tumor Detection Method of the Invention

As previously argued, overexpression of proteins containing domains similar to those of the microbial nucleases has been described in tumor cells. The author of the present invention has shown that specific nucleases such as SND1 and pancreatic cancer cell lysates are capable of activating fluorescence probes and the MRI activatable contrast agent of the invention (Example 3 and 4, respectively), thus showing its utility for image diagnosis of cancer. Thus, in an additional aspect, the present invention relates to an in vitro method for determining whether a subject suffers or not from cancer, particularly from colon cancer or from pancreatic cancer that comprises
(i) contacting a sample from said subject with a MRI activatable contrast agent according to the invention or with a pharmaceutical composition thereof,
(ii) detecting the signal derived from the contrast agent,and
(iii) comparing the signal of the contrast agent detected in (ii) to a reference value,
   wherein
   if the contrast agent shows a signal detectable by MRI higher than a reference value, then the subject suffers from colon cancer or from pancreatic cancer, or
   if the contrast agent shows a signal detectable by MRI similar to or lower than a reference value, then the subject does not suffer from colon cancer or from pancreatic cancer.

In a first step, the in vitro method of the invention for determining whether a subject suffers or not from cancer, particularly from colon cancer or from pancreatic cancer, comprises contacting a sample from said subject with a MRI activatable contrast agent according to the invention or with a pharmaceutical composition thereof.

The sample from the subject suspected to suffer from cancer, particularly from colon cancer or pancreatic cancer, and the MRI activatable contrast agent are put into contact and incubated under conditions suitable for the interaction of the contrast agent with the sample (e.g temperature 37° C., detection in less than 1 hour and a concentration able to differentiate between healthy and disease conditions), so that a signal is detected by MRI if microbial nuclease activity, associated to the tumor, is present in the sample.

Samples according to the invention have been previously mentioned in the context of the method of the invention for detection of microbial infection. Particularly preferred samples include blood samples and biopsy samples, particularly colon biopsy samples and pancreas biopsy samples. Methods for isolating samples are well known to those skilled in the art and have been described previously.

In a second step, the in vitro method of the invention for determining whether a subject suffers cancer, particularly from colon cancer or pancreatic cancer, comprises detecting the signal shown by the contrast agent of the invention. Detection of the signal of the invention has been described previously in the context of the method of the invention for the detection of a microbial infection and incorporated herein. In a particular embodiment, signal intensity is determined, wherein detection of the activated contrast agent by MRI is measured as signal intensity derived from said agent. In a particular alternative embodiment, T1 value is determined, wherein detection of the activated contrast agent is measured as the T1 value derived from said agent.

In a third step, the in vitro method of the invention for determining whether a subject suffers or not from cancer, particularly colon cancer or pancreatic cancer, comprises determining whether the signal detectable by MRI and due to the contrast agent of the invention in the sample from the subject is modified when compared to a reference value, wherein if the contrast agent shows a signal detected by MRI higher than the reference value, then the subject suffers from colon cancer or from pancreatic cancer, and wherein if the contrast agent shows a signal detectable by MRI similar to or lower than the reference value, then the subject does not suffer from colon cancer or from pancreatic cancer.

The terms “high levels”, “low levels” and “similar levels” have been described previously, as well as the term “reference value”. In the context of the in vitro method of the invention for determining whether a subject suffers or not from cancer, particularly colon cancer or pancreatic cancer, the reference value corresponds to the signal detectable by MRI due to the activatable contrast agent of the invention in a control sample, wherein said control sample is a sample obtained from a healthy subject or from a subject not diagnosed with cancer, particularly colon cancer or pancreatic cancer. In an alternative embodiment, the reference value corresponds to the signal detectable by MRI in a control sample, wherein said control sample is a sample obtained from a healthy subject or from a subject not diagnosed with cancer, particularly colon cancer or pancreatic cancer, in the absence of the activatable contrast agent of the invention. The reference value for the signal detectable by MRI may correspond to any of the parameters associated to the activatable contrast agent that can be measured in order to determine if the contrast agent becomes or not activated. In particular, the reference value relates to parameters of a contrast agent including magnetic signal intensity and T1 time. In a more particular embodiment, the reference value relates to magnetic signal intensity or to T1 time value as determined in a control sample obtained from a healthy subject or from a subject not diagnosed with cancer, particularly colon cancer or pancreatic cancer.

In a particular embodiment, detection of the activated contrast agent by MRI is measured as signal intensity derived from said agent, wherein an increased signal intensity derived from said activated agent in comparison to the reference value is indicative of a higher signal derived from said activated agent and, therefore, of activation of the contrast agent related to nuclease activity derived from a tumor, particularly from a colon tumor or a pancreatic tumor.

In a particular alternative embodiment, detection of the activated contrast agent is measured as the T1 value derived from said agent, wherein a decreased T1 value derived from said activated agent in comparison to the reference value is indicative of a higher signal derived from said activated agent and, therefore, of activation of the contrast agent related to nuclease activity derived from a colon tumor or a pancreatic tumor.

### Nuclease Activity Detection Method of the Invention

In another aspect, the invention relates to an in vitro method for detecting nuclease activity in a biological sample that comprises
(i) contacting the sample with a MRI activatable contrast agent according to the invention or with a pharmaceutical composition according to the invention, under suitable conditions for interaction between the sample and the contrast agent,
(ii) detecting the signal derived from the contrast agent, and
(iii) comparing the signal of the contrast agent detected in (ii) to a reference value,
   wherein
if the contrast agent shows a signal detectable by MRI higher than a reference value, then nuclease activity exists in the sample, or
if the contrast agent shows a signal detectable by MRI similar to or lower than a reference value, then nuclease activity does not exist in the sample.

Thus, in a first step, the in vitro nuclease activity detection method comprises contacting the sample with a MRI activatable contrast agent according to the invention, under suitable conditions for interaction between the sample and the contrast agent. Biological samples according to the invention, as well as suitable conditions for interaction between the sample and the contrast agent of the invention, have been described previously and incorporated herein. Particular preferred samples include blood, urine and biopsy samples.

In a particular embodiment of the nuclease activity detection method of the invention, the nuclease activity is derived from a microbial nuclease activity or from a tumor cell. Preferably, microbial nuclease activity is derived from bacterial, viral or fungal nuclease activity. Exemplary, non-limiting examples of bacteria, virus and fungi have been described previously and incorporated herein. In a particular embodiment, bacteria whose nuclease activity is detected are selected from the group consisting of Staphylococcus aureus, Streptococcus pneumoniae, and Mycobacterium tuberculosis. In a particular embodiment, the virus whose nuclease activity is detected is a cytomegalovirus. In a particular embodiment, the fungus whose nuclease activity is detected is Aspergillus. Alternatively, nuclease activity is derived from a colon tumor cell or from a pancreas tumor cell.

In a second step, the in vitro nuclease activity detection method comprises detection derived from the activated contrast agent.

In a particular embodiment, detection of a signal from the activated contrast agent is performed by MRI, as previously described. In an alternative particular embodiment, detection of a signal from the activated contrast agent is performed by nuclear magnetic resonance (NMR), as previously described.

Parameters associated to the signal derived from the contrast agent have been previously described and include, without limitation, signal intensity and T1 time. In a particular embodiment, signal intensity is determined, wherein detection of the activated contrast agent by MRI is measured as signal intensity derived from said agent. In a particular alternative embodiment, T1 value is determined, wherein detection of the activated contrast agent is measured as the T1 value derived from said agent.

In a third step, the in vitro nuclease activity detection method comprises comparing the signal derived from the contrast agent to a reference value, wherein if the contrast agent shows a signal higher than a reference value, then nuclease activity exists in the sample, or if the contrast agent shows a signal similar to or lower than a reference value, then nuclease activity does not exist in the sample. Reference values according to the invention have been previously described and incorporated herein, as well as definitions of the terms “higher than”, “similar to” and “lower than”. The reference value for the signal detectable by MRI may correspond to any of the parameters associated to the activatable contrast agent that can be measured in order to determine if the contrast agent becomes or not activated. In particular, the reference value relates to parameters of a contrast agent including magnetic signal intensity and T1 time. In a more particular embodiment, the reference value relates to magnetic signal intensity or to T1 time value as determined in a control sample wherein nuclease activity is known not to be present.

In a particular embodiment, detection of the activated contrast agent, preferably by MRI, is measured as signal intensity derived from said agent, wherein an increased signal intensity derived from said activated agent in comparison to the reference value is indicative of a higher signal derived from said activated agent and, therefore, of activation of the contrast agent related to nuclease activity. In an alternative embodiment, detection of the activated contrast agent is measured as the T1 value derived from said agent, wherein a decreased T1 value derived from said activated agent in comparison to the reference value is indicative of a higher signal derived from said activated agent and, therefore, of activation of the contrast agent related to nuclease activity. In a preferred embodiment, nuclease activity is derived from microbial infection or from cancer, particularly colon cancer or pancreatic cancer.

In an alternative embodiment, the invention relates to an in vitro method for detecting nuclease activity in a biological sample that comprises
(i) contacting the sample with a MRI activatable contrast agent according to the invention or with a pharmaceutical composition according to the invention, under suitable conditions for interaction between the sample and the contrast agent, and
(ii) determining whether a signal from the activated contrast agent is detected, wherein detection of the activated contrast agent is indicative of nuclease activity in the biological sample, and absence of detection of the activated contrast agent is indicative of absence of nuclease activity in the biological sample.

The first step of contacting the sample with the MRI activatable contrast agent of the invention is performed as previously described.

In a second alternative step, the in vitro nuclease activity detection method comprises determining whether a signal from the activated contrast agent is detected.

Detection methods of the activated contrast agent have been described above. Therefore, if a signal derived from the MRI activatable contrast agent is detected, e.g. the contrast agent becomes activated in the presence of the sample under analysis for nuclease activity and a signal that can be detected is produced, it is indicative of the presence of nuclease activity in said sample under analysis. Otherwise, if no signal is detected derived from the activated contrast agent, i.e. the agent does not become activated, it is indicative of the absence of nuclease activity in said sample under analysis. As previously mentioned, parameters associated to the signal derived from the contrast agent include, without limitation, signal intensity and T1 time.

In a further aspect, the present invention relates as well to a MRI activatable contrast agent as previously described or to a pharmaceutical composition thereof for use in the detection of nuclease activity in a biological sample, wherein the nuclease activity is a microbial nuclease activity or a tumor cell nuclease activity, particularly colon or pancreatic cell nuclease activity. Alternatively, the invention relates to a MRI activatable contrast agent as previously described or to a pharmaceutical composition thereof for use in the diagnosis, in vivo and/or in vitro of a microbial infection or in the diagnosis of cancer, particularly colon cancer or pancreatic cancer in a subject. Alternatively, the invention relates to the use of a MRI activatable contrast agent as previously described or to a pharmaceutical composition thereof in the diagnosis, in vivo and/or in vitro of a microbial infection or in the diagnosis of cancer, particularly in the diagnosis of colon cancer or in the diagnosis of pancreatic cancer in a subject.

The invention is described in detail below by means of the following examples which are to be construed as merely illustrative and not limitative of the scope of the invention.

### EXAMPLES

### Example 1

### Design of the MRI Activatable Probe

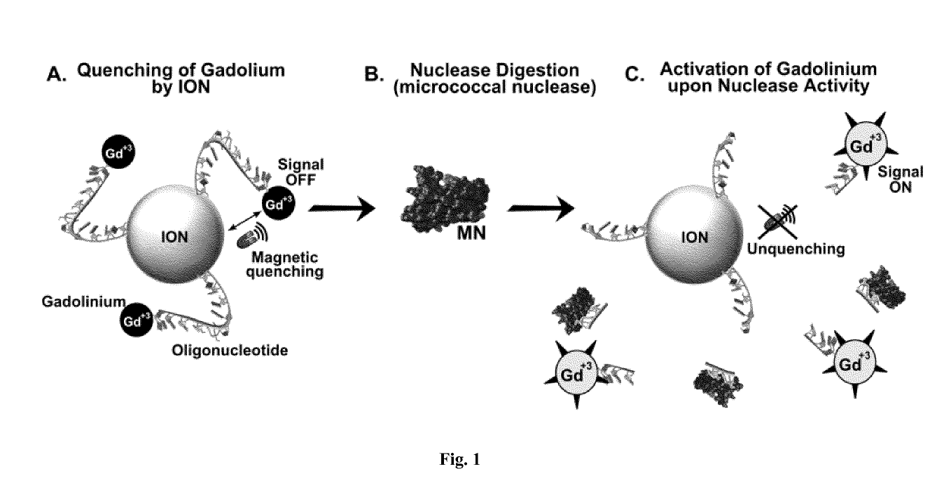

The inventor of the present invention has designed a MRI probe for detecting a microbial endonuclease that comprises a substrate oligonucleotide of 2-30 nucleotides in length, an Iron Oxide Nanoparticle (ION) operably linked to one end of the oligonucleotide, and gadolinium III (Gd) operably linked to the other end of the oligonucleotide. The proximity of ION and Gd, quenches the relaxation properties of Gd (“off” state) and only after digestion of the oligonucleotide by a specific nuclease such as a bacterial nuclease, the Gd diffuses away from the ION and the relaxation properties are recovered (“on” state). The ION and Gd are separated by at least one oligonucleotide-cleavable residue (see FIG. 1).

The MRI-probe has three main components:
(a) Oligonucleotide: The oligonucleotide is synthetized with the standard method (phosphoramidite monomers as building block) and purified by HPLC. Then the oligonucleotide is modified at the 5′-end with a chelator (DTPA) to facilitate the binding to Gadolinium (Gd+3). Additionally, the 3′-end of the oligonucleotide is modified with a thiol group (SH) that allows the conjugation to Iron oxide nanoparticale (ION) by a maleimide coupling chemistry.
(b) Gadolinium: (Gd⁺³): Gadolinium is a paramagnetic molecule that is used as the reported molecule in this probe approach, and it is responsible for the MRI T1 signal upon degradation by nuclease activity.
(c) Iron Oxide Nanoparticle (ION): Superparamagnetic nanoparticle responsible for the quenching by proximity to gadolinium and provides the maleimide coupling group on the surface to attach the thiolyted-oligos. Additionally, ION is responsible for the MRI T2 signal.

1.1.Materials and Methods

### Materials

Oligo 1 (5′-NH2-mCmUmCmGTTmCmGmUmUmC-Biotin-3 ‘) [5 ’-NH2-(SEQ ID NO: 1)-Biotin-3′, Oligo 2 (5′-NH2-TTmCmGmCmUmUmCmGmGmCmGmAmA-Biotin-3′) [5′-NH2-(SEQ ID NO: 2)-Biotin-3′] and Oligo 3 (5′-NH2-mCTAmCmGmCmUmUmCmGmGmCmGTAmG-Biotin-3′ [5′-NH2-(SEQ ID NO: 3)-Biotin-3′], modified at the ends with amine and biotin, where the m stands for 2′O-Methyl nucleotides and TT for DNA thymidines, were purchased from IDT technologies. The bifunctional chelator (p-SCN-Bn-DTPA) was purchased from Macrocyclics. Iron Oxide superparamagnetic nanoparticles were purchased from ADEMTECH SA, Amicon ultra centrifugal filters were purchased from Merkc. Micrococcal nuclease was purchased from Worthington Biochem. Gadolinium III chloride hexahydrate, PBS (phosphate-buffered saline), HEPES (4-(2-hydroxyethyl)piperazine-1-ethanesulfonic acid), calcium chloride, magnesium chloride hexahydate, acetic acid, sodium hydroxide were purchased from Sigma.

### MRI Activatable Contrast Agent Preparation

The three oligonucleotide sequences (Oligo 1 amine-biotin, Oligo 2 amine-biotin and Oligo 3 amine-biotin), were conjugated to p-SCN-Bn-DTPA at a ratio of 1:100 in HEPES buffer pH 8.5 at 4° C. overnight. Then, the conjugate 1 was purified and filtered three times using Amicon ultra centrifugal filter at 1800 g for 40 min at room temperature (RT). Then, conjugate 1 was incubated with Gadolinium chloride hexahydrate at a ratio of 1:1 (DTPA/Gd) at room temperature (RT) for 2 hours. The conjugate 2 was filtered twice using Amicon filters, as described above. Finally, the conjugate 2 (Biotin-Oligo-DTPA-Gd) was incubated with 100 μL of Iron Oxide superparamagnetic nanoparticles for 2 h at 750 rpm at 25° C. The conjugate 3 (named as MRI probe) was purified by magnetic separation and washed three times.

### Relaxation Measurements

NMR measurements at 1.41 T were performed in a relaxometer Minispec MQ60 (Bruker Optik GmbH, Ettlingen, Germany) at 37° C. T1 values were determined by the inversion-recovery method using 8 inversion times (1, 4, 15, 62, 244, 960, 3800 and 15000 ms) one scan and a repetition time of 15 secs. MRI probes in PBS, PBS containing MN and Oligos modified with Gd, but without ION were evaluated to validate the activation of gadolinium (unquenching) by nuclease activity.

1.2.Results

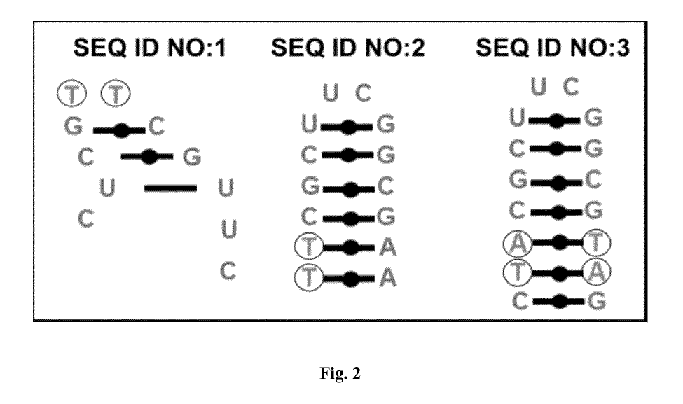

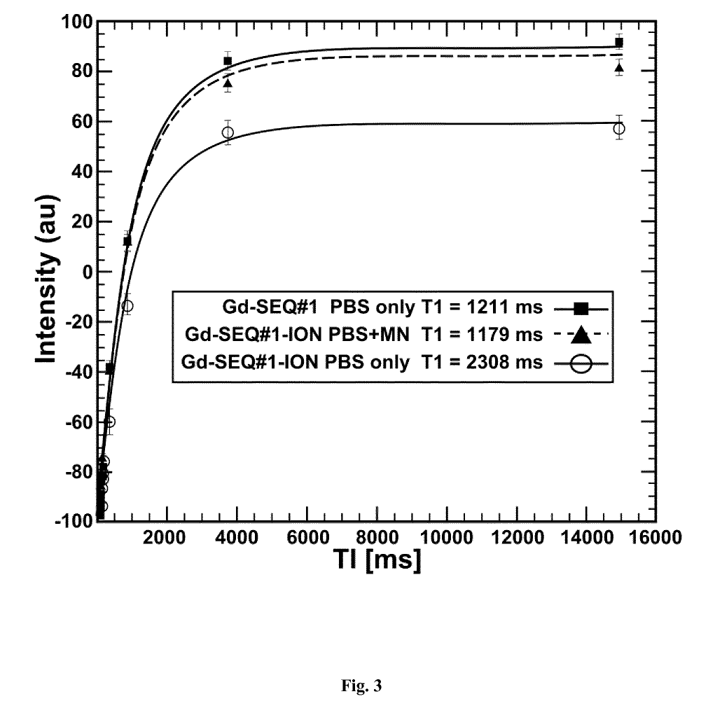

To demonstrate the feasibility of the MRI activatable contrast agents based on nuclease activity, the inventor modified both ends of an oligonucleotide with Gadolinium (Gd) and Iron Oxide Nanoparticle (ION), respectively. The selected oligonucleotides confer specificity to MN by TT SEQ ID NO:1 and 2 and TA by SEQ ID NO:3 sequences, while resistance to endogenous nucleases in mouse and human serum is provided by the 2′-O-Methly nucleotide. 2D structure of the three sequences is provided in FIG. 2. In this setup, the longitudinal spin-lattice magnetic relaxation (T1) of the Gd is quenched by the magnetic field provided by the proximity to ION. However, upon cleavage of the oligonucleotide by the specific nuclease (e.g. MN), the Gd diffuses away from the ION, and thus allowing for the recovery of its magnetic relaxation properties (unquenching) that can be measured by MRI technology.

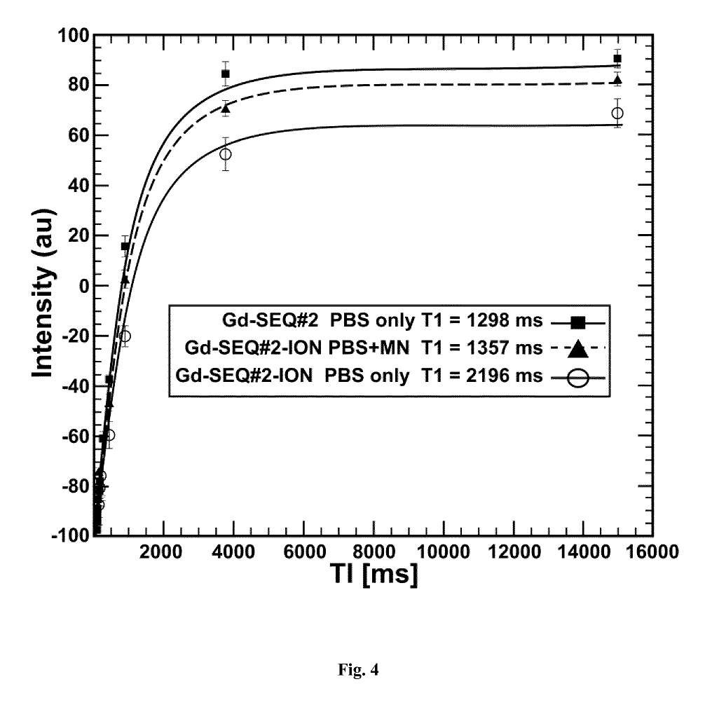

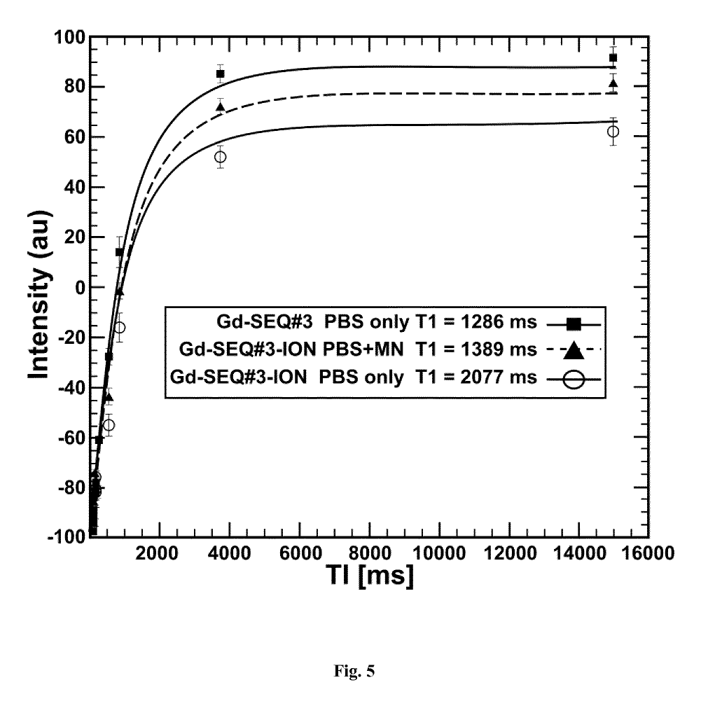

In order to verify the quenching of Gd by the magnetic field of ION, the relaxation properties of the MRI probe conjugates (Gd-oligonucleotide-ION, prepared as described in the Materials and Methods section) were evaluated using Minispec mq60 (Bruker). As shown in FIG. 3, the MRI-SEQ#1 probe (5′-Gd-mCmUmCmGTTmCmGmUmUmC-ION-3′) incubated with PBS only, has reported an efficient quenching of Gd, with a T1 value of 2308 ms. The same MRI-SEQ#1 probe incubated in PBS and supplemented with MN (1U/μL) has shown a T1 value of 1179 ms, indicating specific unquenching based on MN activity. Moreover, samples containing Gd-SEQ#1 conjugate (without ION) have reported a T1 value of 1211 ms, similar to the MRI SEQ#1 probe in the presence of MN, thus confirming the activation of Gd based on nuclease activity. Two additional oligonucleotides (SEQ ID NO: 2 and 3) containing the TT moiety at the beginning of the sequence, and the TA moiety forming a double stranded structure, respectively, were also evaluated. In both cases, the probes MRI-SEQ#2 (FIG. 4) and MRI-SEQ#3 (FIG. 5) have shown similar results to those reported by MRI-SEQ#1 (FIG. 3). These results demonstrate the viability of the concept of MRI activatable probes based on nuclease activity with specific targeting for MN and, hence, their physiological applications. On the other hand, the capability of MN to digest substrates with different structure, such as single and double stranded have been demonstrated as well, and thus suggesting the possibility to design MRI activatable probes based on sequence, chemical modifications and structural features of nucleic acids.

### Example 2

### Dual T1-T2 Activatable MRI Probe

2.1. Materials and Methods

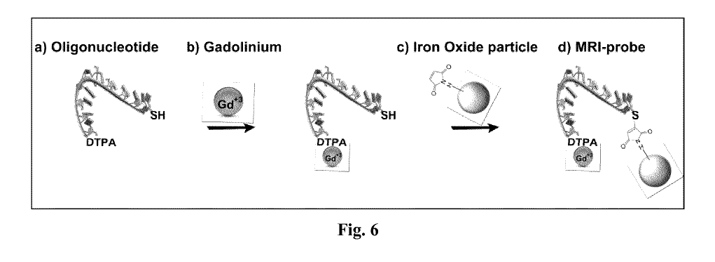

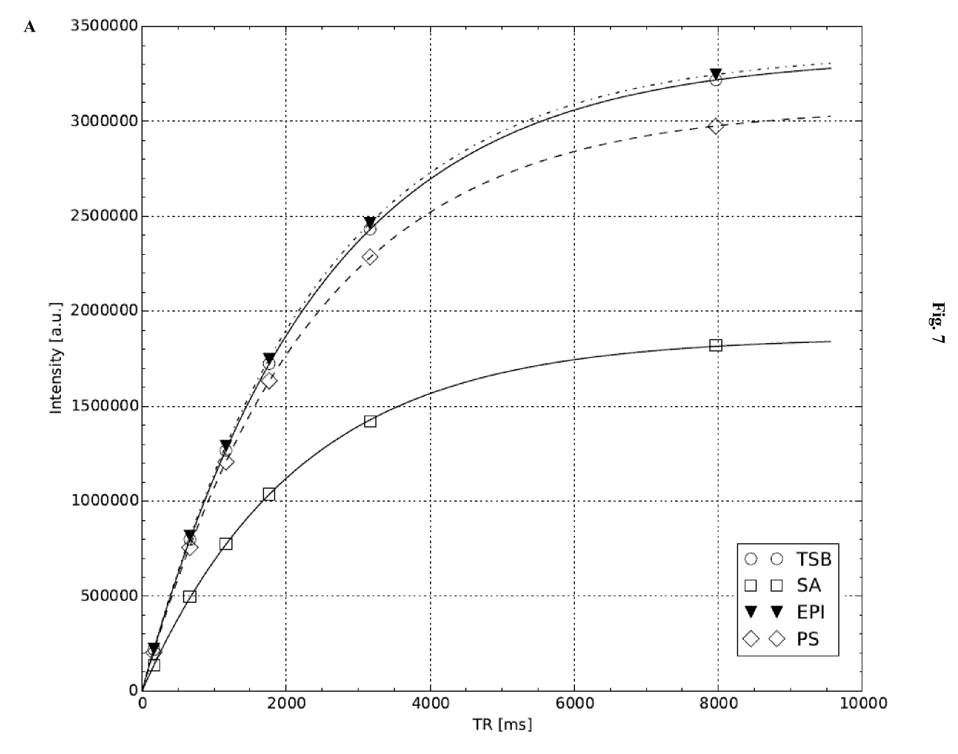

Oligo 4 (5′-Gadolinium-DTPA-mCmUmCmGTTmCmGmUmUmC-SH-3′) (SEQ ID NO: 4) modified at the ends with Gadolimium-DTPA and a thiol group, where the m stands for 2′O-Methyl nucleotides and TT for DNA thymidines, were purchased from Biomers. Iron Oxide superparamagnetic nanoparticles modified with maleimide groups were purchased from Micromods. The modified oligos were couple to the ION by thioester chemistry (FIG. 6). MRI-probe complex was purified and photon correlation spectroscopy (PCS) was used to determine the hydrodynamic particle diameter of the particle samples. The PCS measurements were performed with a Malvern Zetasizer Nano ZS-90 (Malvern Instruments Ltd., Worcestershire, UK). Samples were diluted in 0.22 μm filtered water to an iron concentration of approximately 0.1 mg/ml prior to analysis.

Bacterial cultures supernatant: S. aureus and S. epidermidis bacteria cultures were grown in tryptic soy broth (TSB) for 24h. Next, the sub-cultures (1:500) were grown overnight into 50 ml fresh broth and grown for 24 h at 37 ° C. with shaking at 200 rpm. Then, each culture was centrifuged at 4,000-6,000×g for 10 min and the supernatant was stored for further use. Pig serum and TSB cultures media (obtained from Sigma-Aldrich) were used as controls.

Sample preparation: 1μL of MRI-probe at 3 mg/mL concentration (suspension) was incubated with 299 μL, of bacterial supernatant of S.aureus and S. epidermidis, along with cultures media, and pig serum as controls. All samples were kept at 37° C. and MRI signal recorded after 1 hour of incubation.

### Measurement:

All MRI experiments were performed with a Bruker Biospec 7 T horizontal scanner operating at a proton frequency of 300 MHz (Bruker Biospin GmbH, Ettlingen, Germany) with a 12 cm gradient capable of delivering 400 mT/m and a rise time of 80 ms. 40 mm inner-diameter quadrature volume resonator was used. The aquisition parameters used for T1 and T2 experiments are as follow:

T1-Spin echo saturation recovery (RARE with variable TR) Bruker METHOD=RAREVTR PROTOCOL [′<RARE-T1+T2-map>′] TE effective=15 ms, RARE factor=4 FOV=25×25 mm, ACQ Matrix=128×128, RECO Matrix=128×128 Slice Thickness=3.00 mm, N Slices=3 Nunber of TR=6 TR times=[200.0, 700.0, 1200.0, 1800.0, 3200.0, 8000.0] ms.

T2-weighted multi-spin-echo sequence was measured with the following parameters: Bruker METHOD=MSME PROTOCOL [′<MSME-T2-map>′] TR/TE=2500/12 ms, FOV=25×25 mm, ACQ Matrix=256×256, RECO Matrix=256×256, Slice Thickness=3.00 mm, N Slices=3 N Echoes=16 T Echoes=[12.0, 24.0, 36.0, 48.0, 60.0, 72.0, 84.0, 96.0, 108.0, 120.0, 132.0, 144.0, 156.0, 168.0, 180.0, 192.0] ms.

2.2. Results

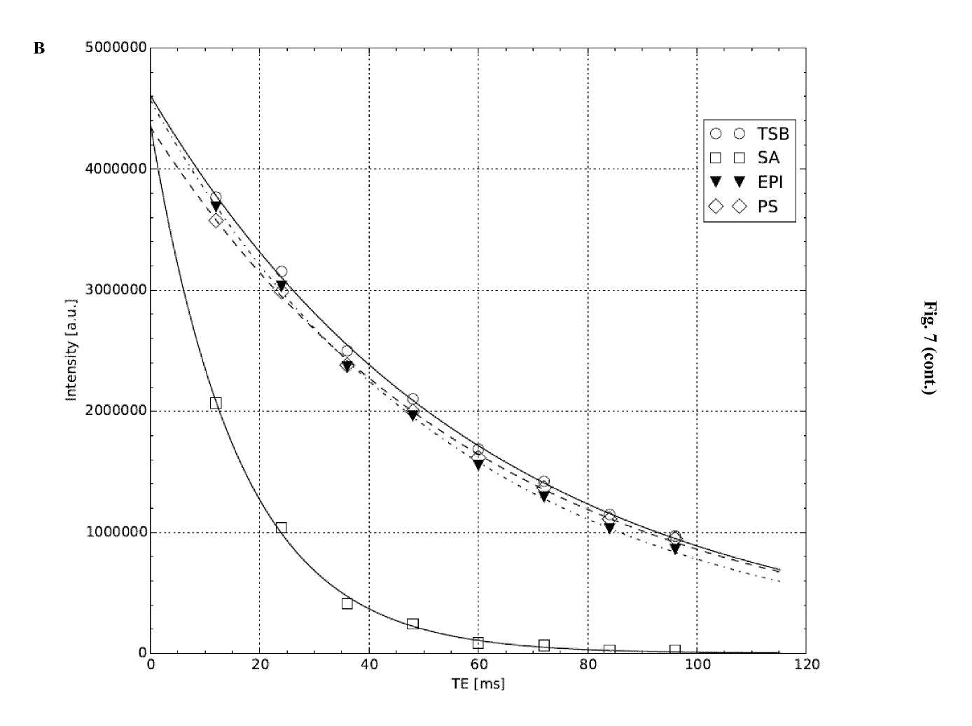

A dual-mode MRI contrast agent (T1-T2) for the specific targeting of S. aureus is reported. As far as the inventors know, this is a novel dual-mode activatable probe reported in the contrast agent field. First, the capability to activate the MRI-probe by S. aureus, which produces MN, and S. epidermis, a genus of Staphylococcus that has been reported to produce undetectable levels of MN, was evaluated. The results have shown that S. aureus cultures were able to activate more efficiently the MRI-probe in both modalities T1 and T2 (FIGS. 7A and 7B). In contrast, the control samples (S. epidermides cultures, TSB culture media and pig serum) have reported a minimal activation of the MRI-probe (Table 1). These results demonstrate the feasibility of a dual-mode MRI contrast agent based on the activatable probe approach. As the main conclusion, the inventors are in the position to claim that the dual MRI-probe will provide more sensitivity and specificity compared to the single mode contrast agents currently available in the market, and this could represent an interesting alternative for diagnostic intervention.

TABLE 1
T1 and T2 activation values for the MRI-probe

| | |
|---|---|
| T1 (ROI mean) | T2 (ROI mean) |

| | | | |
|---|---|---|---|
| TSB | 2440.16 +/− 4.25 | ms | 60.71 +/− 0.86 ms |
| SA | 2175.95 +/− 19.28 | ms | 16.17 +/− 0.53 ms |
| EPI | 2408.66 +/− 4.96 | ms | 56.49 +/− 0.77 ms |
| PS | 2337.95 +/− 10.09 | ms | 61.7 +/− 0.84 ms |

### Example 3

### Activation of the MRI Probe by SND1 Nuclease

3.1. Materials and Methods Relaxation Measurements

1 μL, of AAA chimeric MRI-probes at 3 mg/mL (5′-Galodinium-DTPA-SEQ-ID NO: 10-ION, where SEQ ID NO: 10 is 5′-mUmCmUmCmCmUfAfAfAmUmCmCmUmCmU and where “m” stands for 2′-O-Methyl, synthesized as indicated in example 2) incubated in nuclease buffer containing SND1 (0.082 μg/mL), and 1 μL of AAA chimeric MRI-probe incubated in nuclease buffer only (control) were evaluated by NMR measurements after 1 hour of incubation at 37° C. The NMR measurements at 1.41 T were performed in a relaxometer Minispec MQ60 (BrukerOptikGmbH, Ettlingen, Germany) at 37° C. T1 values were determined by the inversion-recovery method using 8 inversion times (1, 4, 15, 62, 244, 960, 3800 and 15000 ms) one scan and a repetition time of 15 secs.

3.2. Results

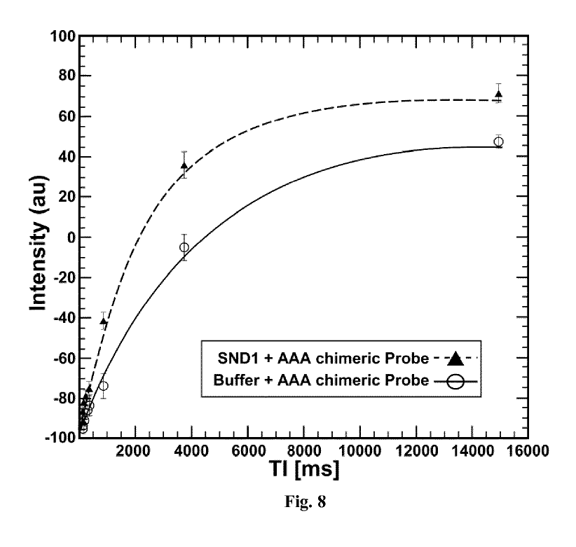

SND1 has been reported as a biomarker that is overexpressed in cancer cells. However, SND1 has been only exploited as ligand for targeting (mostly antibodies), and thus its function as a nuclease has not been used for diagnosis proposes. Herein, the nuclease activity of SND1 is described as an additional feature for targeting cancer cells. In order to prove that the AAA chimeric probe is suitable for MRI detection, the oligonucleotide was synthesized as previously described in example 2 and the relaxivity properties evaluated. The NMR analysis shows that SND1 can active the MRI-AAA chimeric probe (FIG. 8). All together, these results suggest the possibility of targeting cancer cells where SND1 acts as a cancer marker using chimeric oligonucleotides containing 2′-Fluoro and 2′-O-Methyl, or others, that could provide a means to differentiate normal (healthy) cells and cells were SND1 is overexpressed.

### Example 4

### Activation of the MRI Probe by Pancreatic Cancer Cell Lysates

4.1. Materials and Methods

Cell cultures: Pancreatic cancer cells (adenocarcinoma) and healthy cell (epithelial) cells were cultured in Dulbecco's modified Eagle's medium (DMEM) supplemented with 10% fetal bovine serum, 100 U/mL penicillin, and 100 mg/mL streptomycin (Gibco, USA). All cells were maintained at 37° C. in humidified air with 5% CO₂. When the cells reached 80% confluency, they were harvested with 0.05% Trypsin-EDTA, and washed with PBS. Next, cells were peletted at 1500 rpm for 5 min and resuspended in lysis buffer (1% Triton X-100 in 50 mM TrisHCl pH8.5+150 mM NaCl).

Relaxation Measurements

1 μL of All 2′-F MRI-probe (5′-Gadolinium-DTPA-SEQ ID NO: 11-ION, where SEQ ID NO: 11 is 5 ‘-fUfCfUfCfGfUfAfCfGfUfUfC, where “f” stands for 2′-fluoro) at 3 mg/mL (Synthesized as indicated in example 2) incubated in pancreatic cell lysate (sample), and 1 μL of All 2′-F MRI-probe incubated in epithelial (healthy) cells lysate (control cell line), along with 1 μL of All 2′-F MRI-probe incubated in nuclease buffer (background control), were evaluated by NMR measurements after 1 hour of incubation at 37° C. The NMR measurements at 1.41 T were performed in a relaxometer Minispec MQ60 (BrukerOptikGmbH, Ettlingen, Germany) at 37° C. T1 values were determined by the inversion-recovery method using 8 inversion times (1, 4, 15, 62, 244, 960, 3800 and 15000 ms) one scan and a repetition time of 15 secs. 4.2. Results

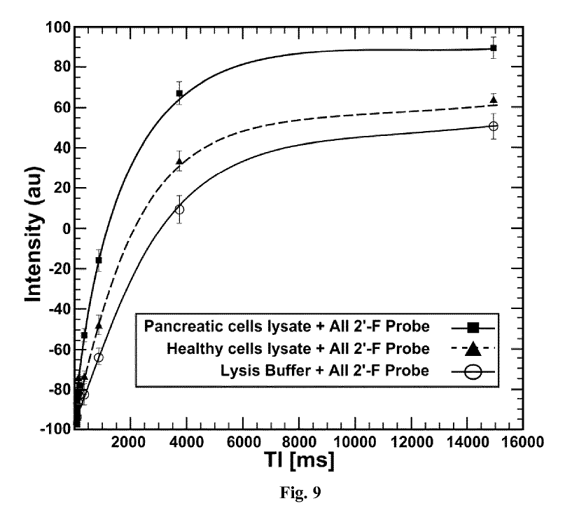

Herein, it is hypothesized that cancer cells can be differentiated from healthy cells based on their nuclease activity, and thus any method based on this property (nuclease degradation activity) represents a novel alternative for diagnostic and therapeutic intervention. In addition, the targeting nuclease can be found extracellular (e.g. secreted), on the cell membrane or located at intracellular compartments (e.g. cytosol, nucleus, mitochondria, etc). As proof of concept study, the nuclease activity of pancreatic adenocarcinoma cell lysates was evaluated using the all 2′-F MRI-probe. Healthy cell lysates along with the lysis buffer was used as a control to thoroughly address the nuclease degradation profile of these cell lines. NMR measurements (FIG. 9B) show that the pancreatic cell lysate was successfully differentiate from healthy cell lysate. In summary, four important findings are reported:

1. Pancreatic cancer cells can be specifically differentiated from healthy cells by using nucleases as biomarkers.

2. Oligonucleotide modifications, such as 2′-Fluoro (and possibly others) represent a promising alternative to design specific targeting probes for pancreatic cancer cells.

3. Herein, the first evidence for targeting cancer cells using chemically-modified oligonucleotides is reported, and this represents an attractive option for intervention, for diagnostics and therapeutics. It is anticipated that other cancer cell lines can be targeted using this probe technology and these nuclease biomarkers can be found as extracellular secreted nucleases, trans-membrane located or at intracellular compartments. 4. Moreover, it is anticipated that nuclease-activated oligonucleotide probes can be selectively digested (activated) by target nucleases also expressed by/in circulating tumor cells (CTCs) from body fluids (blood, urine, cerebral spinal fluid, etc.).

1. An activatable contrast agent for magnetic resonance imaging (MRI) comprising:
(i) a superparamagnetic agent,
(ii) at least one paramagnetic agent, and
(iii) at least one DNA, RNA or DNA-RNA, single or double stranded oligonucleotide, one end of said at least one oligonucleotide operably linked to the superparamagnetic agent in (i) and the other end of said at least one oligonucleotide operably linked to the at least one paramagnetic agent in (ii), wherein said at least one oligonucleotide comprises
at least one region comprising nucleotides conferring resistance to mammalian endonucleases, and
at least one region comprising a nucleotide sequence conferring sensitivity to a microbial nuclease but not to mammalian endonucleases,
and wherein said oligonucleotide in (iii) allows magnetic quenching between the superparamagnetic agent in (i) and the at least one paramagnetic agent in (ii).

2. The contrast agent according to claim 1, wherein the oligonucleotide comprises at least one chemically modified nucleotide selected from the group consisting of 2′-O-methyl-nucleotide, 2′-fluoro-nucleotide, a locked nucleic acid (LNA), an unlocked nucleic acid (UNA), and 2′-deoxy-2′-fluoro-β-d-arabinonucleic acid (FANA).

3. The contrast agent according to the preceding claim, wherein the chemically modified nucleotides are comprised by the region of the oligonucleotide conferring resistance to mammalian endonucleases.

4. The contrast agent according to any of the preceding claims, wherein the region comprising a nucleotide sequence conferring sensitivity to a microbial nuclease but not to mammalian endonucleases comprises a nucleotide sequence selected from the group consisting of TT, AA, TA and AT.

5. The contrast agent according to any of the preceding claims, wherein the quenching between the superparamagnetic agent in (i) and the at least one paramagnetic agent in (ii) is achieved by a length of the oligonucleotide in (iii) of at least 2 nucleotides and/or by the presence of a nucleotide sequence in the oligonucleotide in (iii) having a secondary structure.

6. The contrast agent according to any of the preceding claims, wherein said at least one oligonucleotide comprises the sequence: 5′-mCmUmCmGTTmCmGmUmUmC-3′ (SEQ ID NO:1), 5′-TTmCmGmCmUmUmCmGmGmCmGmAmA-3′ (SEQ ID NO:2), 5′-mCTAmCmGmCmUmUmCmGmGmCmGTAmG-3′ (SEQ ID NO:3), 5′-mUmCmUmCmCmUfAfAfAmUmCmCmUmCmU-3′ (SEQ ID NO: 10) or 5′-fUfCfUfCfGfUfAfCfGfUfUfC-3′ (SEQ ID NO: 11), wherein m represents a 2′O-methyl nucleotide and f represents 2′-fluoro.

7. The contrast agent according to any of the preceding claims, wherein the paramagnetic agent is selected from the group consisting of a gadolinium-based agent and a manganese-based agent, and/or wherein the superparamagnetic agent comprises iron oxide or iron platinum.

8. The contrast agent according to the preceding claim, wherein the gadolinium-based agent is selected from the group consisting of gadolinium-DTPA, gadolinium-DOTA, gadolinium-NOTA, gadolinium-DOTRA, and wherein the manganese-based agent is manganese-DPDP.

9. A pharmaceutical composition comprising an agent according to any of claims 1 to 8.

10. A magnetic resonance imaging (MRI) method for detecting a nuclease activity in a subject, wherein said nuclease activity is a microbial nuclease activity or a tumor cell nuclease activity, that comprises:
(i) administering the activatable contrast agent according to any of claims 1 to 8 or a pharmaceutical composition according to claim 9 to said subject, and
(ii) detecting activated contrast agent by MRI.

11. An in vitro method for determining whether a subject suffers or not from a microbial infection that comprises
(i) contacting a sample from said subject with a contrast agent according to any of claims 1 to 8 or with a pharmaceutical composition according to claim 9,
(ii) detecting the signal derived from the contrast agent, and
(iii) comparing the signal of the contrast agent detected in (ii) to a reference value,
wherein
if the contrast agent shows a signal detectable by MRI higher than a reference value, then the subject suffers from a microbial infection, or
if the contrast agent shows a signal detectable by MRI similar to or lower than a reference value, then the subject does not suffer from a microbial infection.

12. The method according to claim 10 wherein the microbial nuclease activity is due to, or the method according to claim 11 wherein the microbial infection is caused by, a microorganism selected from the group consisting of Staphylococcus aureus, Streptococcus pneumoniae, Mycobacterium tuberculosis, a cytomegalovirus and Aspergillus.

13. An in vitro method for determining whether a subject suffers or not from cancer that comprises
(i) contacting a sample from said subject with a contrast agent according to any of claims 1 to 8 or with a pharmaceutical composition according to claim 9,
(ii) detecting the signal derived from the contrast agent, and
(iii) comparing the signal of the contrast agent detected in (ii) to a reference value,
wherein
if the contrast agent shows a signal detectable by MRI higher than a reference value, then the subject suffers from colon cancer or from pancreatic cancer, or
if the contrast agent shows a signal detectable by MRI similar to or lower than a reference value, then the subject does not suffer from colon cancer or from pancreatic cancer.

14. The in vitro method according to claim 13 wherein the cancer is pancreatic cancer or colon cancer.

15. An in vitro method for detecting nuclease activity in a biological sample that comprises
(i) contacting the sample with a MRI activatable contrast agent according to any of claims 1 to 8 or with a pharmaceutical composition according to claim 9, under suitable conditions for interaction between the sample and the contrast agent,
(ii) detecting the signal derived from the contrast agent, and
(iii) comparing the signal of the contrast agent detected in (ii) to a reference value,
wherein
if the contrast agent shows a signal detectable by MRI higher than a reference value, then nuclease activity exists in the sample, or
if the contrast agent shows a signal detectable by MRI similar to or lower than a reference value, then nuclease activity does not exist in the sample,
or that, alternatively, comprises
(i′) contacting the sample with a MRI activatable contrast agent according to any of claims 1 to 8 or with a pharmaceutical composition according to claim 9, under suitable conditions for interaction between the sample and the contrast agent, and
(ii′) determining whether a signal from the activated contrast agent is detected,
wherein detection of the activated contrast agent is indicative of nuclease activity in the biological sample, and absence of detection of the activated contrast agent is indicative of absence of nuclease activity in the biological sample.

16. The method according to the preceding claim, wherein the nuclease activity in the sample is a microbial nuclease activity or a tumor cell nuclease activity.


SEQUENCE LISTING (11 sequences)

SEQ ID NO: 1 - DNA, 11 bp, Artificial Sequence
  Oligonucleotide 1
  variation/unsure/modified_base (1)..(1): /note=2′O-methyl cytosine
  misc_feature (1)..(4): n is a, c, g, t or u
  variation/unsure/modified_base (2)..(2): /note=2′O-methyl uracile
  variation/unsure/modified_base (3)..(3): /note=2′O-methyl cytosine
  variation/unsure/modified_base (4)..(4): /note=2′O-methyl guanine
  variation/unsure/modified_base (7)..(7): /note=2′O-methyl cytosine
  misc_feature (7)..(11): n is a, c, g, t or u
  variation/unsure/modified_base (8)..(8): /note=2′O-methyl guanine
  variation/unsure/modified_base (9)..(9): /note=2′O-methyl uracile
  variation/unsure/modified_base (10)..(10): /note=2′O-methyl uracile
  variation/unsure/modified_base (11)..(11): /note=2′O-methyl cytosine
nnnnttnnnnn

SEQ ID NO: 2 - DNA, 13 bp, Artificial Sequence
  Oligonucleotide 2
  variation/unsure/modified_base (3)..(3): /note=2′O-methyl cytosine
  misc_feature (3)..(11): n is a, c, g, t or u
  variation/unsure/modified_base (4)..(4): /note=2′O-methyl guanine
  variation/unsure/modified_base (5)..(5): /note=2′O-methyl cytosine
  variation/unsure/modified_base (6)..(6): /note=2′O-methyl uracile
  variation/unsure/modified_base (7)..(7): /note=2′O-methyl uracile
  variation/unsure/modified_base (8)..(8): /note=2′O-methyl cytosine
  variation/unsure/modified_base (9)..(9): /note=2′O-methyl guanine
  variation/unsure/modified_base (10)..(10): /note=2′O-methyl guanine
  variation/unsure/modified_base (11)..(11): /note=2′O-methyl cytosine
  variation/unsure/modified_base (12)..(12): /note=2′O-methyl guanine
ttnnnnnnnnnaa

SEQ ID NO: 3 - DNA, 16 bp, Artificial Sequence
  Oligonucleotide 3
  variation/unsure/modified_base (1)..(1): /note=2′O-methyl cytosine
  misc_feature (1)..(1): n is a, c, g, t or u
  variation/unsure/modified_base (4)..(4): /note=2′O-methyl cytosine
  misc_feature (4)..(13): n is a, c, g, t or u
  variation/unsure/modified_base (5)..(5): /note=2′O-methyl guanine
  variation/unsure/modified_base (6)..(6): /note=2′O-methyl cytosine
  variation/unsure/modified_base (7)..(7): /note=2′O-methyl uracile
  variation/unsure/modified_base (8)..(8): /note=2′O-methyl uracile
  variation/unsure/modified_base (9)..(9): /note=2′O-methyl cytosine
  variation/unsure/modified_base (10)..(10): /note=2′O-methyl guanine
  variation/unsure/modified_base (11)..(11): /note=2′O-methyl guanine
  variation/unsure/modified_base (12)..(12): /note=2′O-methyl cytosine
  variation/unsure/modified_base (16)..(16): /note=2′O-methyl guanine
  misc_feature (16)..(16): n is a, c, g, t or u
ntannnnnnnnnntan

SEQ ID NO: 4 - DNA, 11 bp, Artificial Sequence
  oligonucleotide
  variation (1)..(1): /note=2′O-methyl cytosine
  misc_feature (1)..(4): n is a, c, g, t or u
  variation (2)..(2): /note=2′O-methyl uracile
  variation (3)..(3): /note=2′O-methyl cytosine
  variation (4)..(4): /note=2′O-methyl guanine
  variation (7)..(7): /note=2′O-methyl cytosine
  misc_feature (7)..(11): n is a, c, g, t or u
  variation (8)..(8): /note=2′O-methyl guanine
  variation (9)..(10): /note=2′O-methyl uracile
  variation (11)..(11): /note=2′O-methyl cytosine
nnnnttnnnnn

SEQ ID NO: 5 - DNA, 16 bp, Artificial Sequence
  oligonucleotide
  variation (1)..(1): /note=2′O-methyl cytosine
  misc_feature (1)..(4): n is a, c, g, t or u
  variation (2)..(2): /note=2′O-methyl uracile
  variation (3)..(3): /note=2′O-methyl cytosine
  variation (4)..(4): /note=2′O-methyl guanine
  variation (7)..(7): /note=2′O-methyl cytosine
  misc_feature (7)..(16): n is a, c, g, t or u
  variation (8)..(8): /note=2′O-methyl guanine
  variation (9)..(10): /note=2′O-methyl uracile
  variation (11)..(11): /note=2′O-methyl cytosine
  variation (12)..(16): /note=2′O-methyl uracile
nnnnttnnnnnnnnnn

SEQ ID NO: 6 - DNA, 36 bp, Artificial Sequence
  oligonucleotide
  variation (1)..(1): /note=2′O-methyl cytosine
  misc_feature (1)..(4): n is a, c, g, t or u
  variation (2)..(2): /note=2′O-methyl uracile
  variation (3)..(3): /note=2′O-methyl cytosine
  variation (4)..(4): /note=2′O-methyl guanine
  variation (7)..(7): /note=2′O-methyl cytosine
  misc_feature (7)..(36): n is a, c, g, t or u
  variation (8)..(8): /note=2′O-methyl guanine
  variation (9)..(10): /note=2′O-methyl uracile
  variation (11)..(11): /note=2′O-methyl cytosine
  variation (12)..(36): /note=2′O-methyl uracile
nnnnttnnnnnnnnnnnnnnnnnnnnnnnnnnnnnn

SEQ ID NO: 7 - DNA, 61 bp, Artificial Sequence
  oligonucleotide
  variation (1)..(1): /note=2′O-methyl cytosine
  misc_feature (1)..(4): n is a, c, g, t or u
  variation (2)..(2): /note=2′O-methyl uracile
  variation (3)..(3): /note=2′O-methyl cytosine
  variation (4)..(4): /note=2′O-methyl guanine
  variation (7)..(7): /note=2′O-methyl cytosine
  misc_feature (7)..(61): n is a, c, g, t or u
  variation (8)..(8): /note=2′O-methyl guanine
  variation (9)..(10): /note=2′O-methyl uracile
  variation (11)..(11): /note=2′O-methyl cytosine
  variation (12)..(61): /note=2′O-methyl uracile
nnnnttnnnnnnnnnnnnnnnnnnnnnnnnnnnnnnnnnnnnnnnnnnnnnnnnnnnnnnn

SEQ ID NO: 8 - DNA, 11 bp, Artificial Sequence
  oligonucleotide
  variation (1)..(1): /note=2′O-methyl cytosine
  misc_feature (1)..(11): n is a, c, g, t or u
  variation (2)..(2): /note=2′O-methyl uracile
  variation (3)..(3): /note=2′O-methyl cytosine
  variation (4)..(6): /note=2′O-methyl guanine
  variation (7)..(7): /note=2′O-methyl citosine
  variation (8)..(8): /note=2′O-methyl guanine
  variation (9)..(10): /note=2′O-methyl uracile
  variation (11)..(11): /note=2′O-methyl cytosine
nnnnnnnnnnn

SEQ ID NO: 9 - DNA, 14 bp, Artificial Sequence
  oligonucleotide
  variation (3)..(3): /note=2′O-methyl cytosine
  misc_feature (3)..(14): n is a, c, g, t or u
  variation (4)..(4): /note=2′O-methyl guanine
  variation (5)..(5): /note=2′O-methyl cytosine
  variation (6)..(7): /note=2′O-methyl uracile
  variation (8)..(8): /note=2′O-methyl cytosine
  variation (9)..(10): /note=2′O-methyl guanine
  variation (11)..(11): /note=2′O-methyl cytosine
  variation (12)..(12): /note=2′O-methyl guanine
  variation (13)..(14): /note=2′O-methyl adenine
ttnnnnnnnnnnnn

SEQ ID NO: 10 - DNA, 15 bp, Artificial Sequence
  olignoucleotide
  variation (1)..(1): /note=2′O-methyl uracile
  misc_feature (1)..(6): n is a, c, g, t or u
  variation (2)..(2): /note=2′O-methyl cytosine
  variation (3)..(3): /note=2′O-methyl uracile
  variation (4)..(5): /note=2′O-methyl cytosine
  variation (6)..(6): /note=2′O-methyl uracile
  misc_feature (7)..(9): 2′ fluoro
  variation (10)..(10): /note=2′O-methyl uracile
  misc_feature (10)..(15): n is a, c, g, t or u
  variation (11)..(12): /note=2′O-methyl cytosine
  variation (13)..(13): /note=2′O-methyl uracile
  variation (14)..(14): /note=2′O-methyl cytosine
  variation (15)..(15): /note=2′O-methyl uracile
nnnnnnaaannnnnn

SEQ ID NO: 11 - DNA, 12 bp, Artificial Sequence
  oligonucleotide
  misc_feature: 2′ fluoro
ucucguacguuc